SEQUENCE LISTING (24 sequences)

SEQ ID NO: 1 - DNA, 27 bp, Artificial Sequence
  CDO1 forward primer
acgttttttttcgttttattttcgtcg

SEQ ID NO: 2 - DNA, 23 bp, Artificial Sequence
  CDO1 reverse primer
tcctccgaccctttttatctacg

SEQ ID NO: 3 - DNA, 23 bp, Artificial Sequence
  CDO1 probe
gtggttcgcgacgttgggacgta

SEQ ID NO: 4 - DNA, 23 bp, Artificial Sequence
  PTGER4 forward primer
ttattttcggggttaattcgttc

SEQ ID NO: 5 - DNA, 21 bp, Artificial Sequence
  PTGER4 reverse primer
ataaacatcaccgccgaaata

SEQ ID NO: 6 - DNA, 31 bp, Artificial Sequence
  PTGER4 probe
ttgagtttcgatcggttgaatagtttagtga

SEQ ID NO: 7 - DNA, 19 bp, Artificial Sequence
  HOXA9 forward primer
tatcgcgggtgtaggtttt

SEQ ID NO: 8 - DNA, 26 bp, Artificial Sequence
  HOXA9 reverse primer
aaacgaatttaaaaattacccgaacg

SEQ ID NO: 9 - DNA, 27 bp, Artificial Sequence
  HOXA9 probe
atagttattcgtttggagcgaggggcg

SEQ ID NO: 10 - DNA, 22 bp, Artificial Sequence
  SHOX2 forward primer
aatcgaggatcgcgaatatttc

SEQ ID NO: 11 - DNA, 21 bp, Artificial Sequence
  SHOX2 reverse primer
tcgtaaaaacgcaaactcgac

SEQ ID NO: 12 - DNA, 36 bp, Artificial Sequence
  SHOX2 probe
ttgaatttgttgatttttgtgggttaagcgtttagg

SEQ ID NO: 13 - DNA, 23 bp, Artificial Sequence
  SP9 forward primer
tatttaggttgagtttagtcggc

SEQ ID NO: 14 - DNA, 23 bp, Artificial Sequence
  SP9 reverse primer
cgaaaaaactacttaaatcccgc

SEQ ID NO: 15 - DNA, 25 bp, Artificial Sequence
  SP9 probe
agtttggagttcgttcggtacgagc

SEQ ID NO: 16 - DNA, 22 bp, Artificial Sequence
  ZNF781 forward primer
gtcgttggtataagttgcgttc

SEQ ID NO: 17 - DNA, 23 bp, Artificial Sequence
  ZNF781 reverse primer
aaactataacccgataaatccgc

SEQ ID NO: 18 - DNA, 28 bp, Artificial Sequence
  ZNF781 probe
agacgtgggagcgtttttttgtttttcg

SEQ ID NO: 19 - DNA, 1000 bp, Homo sapiens
cgtccatccataatgagaaggaagaaatctacacttgcatttcttccacctttcacatctttctcatttaaatcctactgtacttgatttgtgttgttcccaacgtactaggtgctatgcatgacgtagaaacgtgtttccttatatgtatgtactagcaggctccttcaagcacatgcctaaggttgaaagattcaaatataattatagcctcttaaattattgcgtcagagcccgccctatcccctcagaaaaagcccccttttacaccatcaaatgaaatatcaggttatttgcactggatattttttaaaagaaaatatttgattttaaaatcaaagagttcttggttttccatcatttcactaattgtcttactagagacccgagtctcctttgtcaaaatatatttcagtacgcattttaaaaaatatgtgtgtgtgtatacttaaatatatatatatataaagtcttggacttattttcacctagcctggggcatatcgtggatagtttcaatcgacaaaacaggcactctctggcctaggagtggaatccatcctcaattttcgcaggctcctcacaattctctatttggaagaagtgtccctctccttcccttttcttttcctcctttactcagcgtcagtccccgcagccatctcctccgaccctttttgtctacgtcccagcgtcgcgaaccacagcggcggaggtggagcggggagaggcgttaggccgggcggctaaaacgcgccgttaaagtgggggagagattgcgcggagcccacgcgatccctgggacgccggagacaacggggctcttgggaaggcgcggagcccggggaagccggggatgtgcgcgtgagccgtgcccgcagggtctccccgcctccgccacctttcttgggtggctctccgcctcgtcctccctccgagggccgttggtacattcctagtgactccaagcgcttaaaaggggcccgggaggatgaacccc

SEQ ID NO: 20 - DNA, 1002 bp, Homo sapiens
ccctcgccgaagagagccaagaagggaagagcgcgctctccaaattgcttttgtaacttgttttcagtgagcattttattgattcagaatctatcgagaatagcactagcgagctacttttcccttgagatgggtcttattcatcttggcaatggagtgagttggattgtggggaggaagaggaatgggaaaatcagtttataaatattaatgtcagcaagagtgtgctgttggcaggacgtatcgcgagcctggagattttggtggccgcagttggtaagtggctacaatccagaaagtaggatcgagttgctccccttgtcttatcagtgtatcgtttctcgggcgcgggtctaacaccttacaagtggtaatttccgctcacggcagctttgtctctcttctaccatccccagacccagccttgcactccaaggctgcgcaccgccagccactatcatgtccactcccggggtcaattcgtccgcctccttgagccccgaccggctgaacagcccagtgaccatcccggcggtgatgttcatcttcggggtggtgggcaacctggtggccatcgtggtgctgtgcaagtcgcgcaaggagcagaaggagacgaccttctacacgctggtatgtgggctggctgtcaccgacctgttgggcactttgttggtgagcccggtgaccatcgccacgtacatgaagggccaatggcccgggggccagccgctgtgcgagtacagcaccttcattctgctcttcttcagcctgtccggcctcagcatcatctgcgccatgagtgtcgagcgctacctggccatcaaccatgcctatttctacagccactacgtggacaagcgattggcgggcctcacgctctttgcagtctatgcgtccaacgtgctcttttgcgcgctgcccaacatgggtctcggtagctcgcggctgcagtacccagacacctggtgcttcatcgactggaccaccaacgtgacggcgcac

SEQ ID NO: 21 - DNA, 1155 bp, Homo sapiens
ggtcctcgtagcaaaggcgaatttaagggttgcccgggcgcccctcgctccaggcgggtagctgtggggacctacacccgcggtactccctgagcggccggtccctgcctggagtgccctggtagggccggcggcggctccgtttgggacggatcctgcgttgaatttgacttttcgagggcggccgcgggtaaactcgcctctcccggggaccgcagggattatttacagggagctcgccaaccaaacacaacagtctaacctttccaagtcctcgtaaatttttacagctgggagccacggcgaggcaaacgaatctgttggtcgtttccgacttcccgccagcctgtgtggcttctgaaacaataactccttatgaaatatcataaatatagatttaaatacagtagagcgacaatgcgatttggctgcttttttatggcttcaattattgtctaattttatgtgaggggctccgctggccgcactcgcacgcgggacccgcgccttcttgatggcgtgattaattgtgatataaaatagtccgcttaagaagtgtgtgtatggggggggagacgggagagtacagagacaaggctagatttgatcttttaatcgtcgttggccacaattaaaacaaaccccatcgtagagcggcacgatccctttacataaaaacatatggcttttgctataaaaattatgactgcaaaacatcggaccattaatagcgtgcggagtgatttacgcgttattgttctgctggacgggcacgtgacgcgcacggccaatgggggcgcgggcgccggcaacttattaggtgactgtacttcccccccggtgccaccaagttgttacatgaaatctgcagtttcataatttccgtgggtcgggccgggcgggccaggcgctgggcacggtgatggccaccactggggccctgggcaactactacgtggactcgttcctgctgggcgccgacgccgcggatgagctgagcgttggccgctatgcgccggggaccctgggccagcctccccggcaggcggcgacgctggccgagcaccccgacttcagcccgtgcagcttccagtccaaggcgacggtgtttggcgcctcgtggaacccagtgcacgcggcgggcgccaacgctgt

SEQ ID NO: 22 - DNA, 1002 bp, Homo sapiens
gttcgccagcgcgcctgacggcgaggttcaggtctgaacccccggcgggcgcagttcctgggattgttgatttccaagctcagggcaagtattaatttctctgcagaccggctcttccggaaatgatttttaacacccatgctggcctggatcctgcgctgggaagaactgcaaacagcctcgactatctaaactcagctttgacttcttaaaacccggagtctctgattcgggcgcagaggataaaggttccagggggatatatatatggtggctgtatatgctcacactagaagagctcctgtttttaaagggaggagaaggacaggaaagtggcaacggatgtgtgcaaacccagagcagaaaaattcatattttattaaacatccgagctgctggttgcatgaaaaggctttgagcaaccaatagaaaccgaggatcgcgaatattccgcttgaacctgttgatctctgtgggttaagcgtttagggtcgagtctgcgtttccacgagggagggcgaagagagaacagaaagagcggttgctttcgcccgccaccggaggctggttttccgcctcctgccttctggcccggcttgggcggcgagccctttgggcagccaacatggcgtgggcgcctgtgctcgtgcgaccccggtcgggcaggcgggacggagattacctggctgtccaggggaccttatgcagggtttggcccgagcccaggggcagcgaggggcgtctgcggatgcggctccctgtgcggcacaacaccggggtctgttgctctcgctgatacctttcggcccaattgttcctaaattagaccagcaagtttgcggtaggcagagagccagcggtggtgagagctggcctgagaacgagccagccttctgggaatggccggtgtgagaggccagtgggtggccttgccgcgcccttatgtccagactttgctctgtgagaggcctggcgctgcgcctttgatttctgtgagtgctg

SEQ ID NO: 23 - DNA, 1002 bp, Homo sapiens
atacaagctgaagttgtgttctgtaggcaaaagcctggacacacaaagtgagggctcacatttccgtctccacttgggccctgctggtgcgggctgtgtctgggtgtctcctcccctcctgcctcctttcctacaagtggggagtcgagtaggactgtcagccctaacttgcaacatctcggggtggtttgtgtgtgggggggtgcttgtggctcggtttcattggtacttaaacctggcttggtgcgtgagtgtaaatttttaattgcactgtaatttcttcagtccccagcccgccctcgccccccccccgcaagtatcccccactactcccccccacccccgcggccctagctagctgacttgactggcacgcgccgggagcccgggctcgggcccctcggagcgtctgattggctgcggggcagctccggtctgctctgcctgcgccctcattgggcgagaggcgcagccagcggcacttcaaagcgggtgctcctcgcacttaggctgagtttagccggcgggagcctggagtccgctcggcacgagcgcggggacgcgggagccgcgcgggacccaagcagtttttccgagcagccgccaggctcagccccgctcccagcctcgctgcgcagccagagacctgctatggccacgtctatactcggggtaagtcgcaagcgcggcaacgcatttgcttgttttaaccggagtaatttttcgttatggcttctggggtctgcggctccggagaaactgttgctgctagactgcagcttcagcttctattagcactttccacatttctgggactgatttttccccaagtttttggaacccagaaagatgcctttgcaagaaaaggccttcttaatcaagtcctttttcatttggtttcaaaaaaaagctagaggtttcttttcttcccctcgtgatttataccccatcccccgcattgctttggggattttgttgcaattatgcgacaatggtgtttcc

SEQ ID NO: 24 - DNA, 1002 bp, Homo sapiens
agctaattttttgtattttagtaaagacggggtttcaccatgttgcccaggctggtctcgaactcctgagctcgggcaatccgcctgcctcggcctcccaaagtgctaggattacaggcgtgagccattgcccccggcctcctcatctttaaattcacaaaaagattgaatatgaagaaaataaaagcctgtgtatttagcactaaaaaaaaaaaagaaaatataataaaatggcagctttaaatctatcagtggctatattaaatgtaaataaattaaatactccagttaaaagagatagacgttcggggtgtatttaaaagccacagacacacatggtcttccgactgccctattcacatgctcagcccctgacagcactggctctgggactaagcccctcgggcagccctgagcctcccagggctgagcacctcgctttgagcagctccgctaccgcgtcctccagtcgggaggcggctgcgttcctgcgcgaagcacgtcggccgcgaaggcctctgggaaatgtattccgggctaccagcccacaaacgcggcggaaccggttgcggggaggttgcttgaattctctcagctgcgccccctgacctagtagtcgctggcacaagctgcgcccggagacgtgggagcgttctcttgttttccgagtgcgcggactcatcgggtcacagtttatgcttttatgacgcggtgagtccagccactgattcctaacggtttagagtggggaggttacgcttgggcgtcctggacgctggcccaagtggtggctgtagaagacgcacggatcccgggatggggctggaggtttgcaagagtccatccgaggggattctggctgcgagtgtgacagagtatatgaaagccgcatggaggatctgattgtagtgtgagcgagagtgagtgcccgcgagggatcgtcacctgaatgaaagtctcggattcaccgcctcttatcttcccttcttcgactgtaaaatg

(19) United States
(12) Patent Application Publication
(10) Pub. No.: US 2025/0361563 A1
(43) Pub. Date: Nov. 27, 2025

(54) METHODS, COMPOSITIONS, AND KITS FOR DETECTING MALIGNANT LUNG NODULES AND LUNG CANCER

(71) Applicant: SUZHOU HUHU HEALTH TECHNOLOGY CO., LTD, Suzhou (CN)

(72) Inventors: Dan LI, Suzhou (CN); Nan JIANG, Suzhou (CN)

(21) Appl. No.: 18/872,833

(22) PCT Filed: Jun. 10, 2022
(86) PCT No.: PCT/CN2022/098197

§ 371 Date: Dec. 9, 2024

(51) Int. Cl. C12Q 1/6886

(52) U.S. Cl. CPC C12Q 1/6886; C12Q 2600/154; C12Q 2600/16

(57) ABSTRACT

The present disclosure provides minimally invasive methods for determining whether a lung nodule is malignant or not by measuring the methylation level of a combination of genes, including CDO1, PTGER4, HOXA9, SHOX2, SP9, and ZNF781. The disclosed methods are also able to detect lung cancer. The present disclosure also discloses polynucleotides and kits that could be used in measuring the methylation level of the CDO1, PTGER4, HOXA9, SHOX2, SP9, and ZNF781.

### FIELD OF THE DISCLOSURE

The present disclosure relates to minimally invasive methods for identifying a malignant lung nodule by measuring the methylation level of a combination of genes, including CDO1, PTGER4, HOXA9, SHOX2, SP9, and ZNF781 in a sample from a subject. The disclosed methods are also able to detect lung cancer. The present disclosure also relates to polynucleotides and kits for use in measuring the methylation level of the CDO1, PTGER4, HOXA9, SHOX2, SP9, and ZNF781.

### SUMMARY

Lung cancer is the leading cause of cancer-related mortality globally. Effective and efficient differentiation of malignant from benign lung nodules using minimally invasive methods is a major unmet clinical need. If such differentiation can be done when the malignant lung nodules are small, such methods can also be used for the early detection of lung cancer. Computer tomography (CT) is commonly used for the detection of lung cancer. However, CT has a high false positive rate. It also requires the doctor to be well-trained and experienced to identify and differentiate malignant lung nodules from benign lung nodules using CT. In addition, CT is not easily accessible and affordable for high-risk populations in developing countries.

Several other approaches including the detection of gene mutations and whole genome sequencing have been studied for the early detection of malignant lung nodules and lung cancer. However, the results and effects of these existing methods are not satisfactory. A recent study demonstrated that the sensitivity of detecting stage I lung cancer using existing methods is less than 22% (Klein et al., 2021). In addition, large panels of next generation sequencing are required in the existing methods, which significantly increase the cost of the tests. Moreover, many existing methods are invasive, requiring lung tissue samples to carry out the assay. Thus, a cost-efficient and minimally invasive test that has high accuracy and specificity is needed for the early detection of malignant lung nodules and the early detection of lung cancer.

The present disclosure provides minimally invasive methods for determining whether a lung nodule is malignant by measuring the methylation level of a combination of genes selected from CDO1, PTGER4, and HOXA9 in a sample from a subject. The methods can also be used to detect whether a subject has at least one malignant lung nodule and/or lung cancer. The present disclosure also provides polynucleotides and kits for use in measuring the methylation level of CDO1, PTGER4, and HOXA9.

DNA methylation is a promising marker for the early detection of cancer because of its stability and heritability. Aberrant DNA methylation results in dysregulation of various genes and occurs in all stages of lung cancer, including initiation, growth, and metastasis. The present disclosure identified six genes that are differentially methylated from subjects with malignant lung nodules, compared to normal lung tissue. The present disclosure showed that detection of hypermethylation of a combination of at least two of the six genes disclosed herein indicates the presence of a malignant lung nodule and enables the early diagnosis of lung cancer. In some embodiments, the methylation level of a combination of CDO1 and PTGER4 is sufficient to distinguish malignant lung nodules from benign nodules. The cost-efficient and minimally invasive methods provided herein are suitable for both malignant lung nodule differentiation and lung cancer diagnosis.

In an aspect, the present disclosure provides a method comprising (a) measuring the methylation level of at least two genes in a sample from a subject, wherein the at least two genes are selected from CDO1, PTGER4, HOXA9, SHOX2, SP9, and ZNF781, and (b) determining if the at least two genes are hypermethylated, wherein detection of hypermethylation of the at least two genes indicates that the subject has at least one malignant lung nodule and/or lung cancer.

In an aspect, the present disclosure provides a method comprising (a) measuring the methylation level of at least two genes in a sample from a subject, wherein the at least two genes are selected from CDO1, PTGER4, HOXA9, SHOX2, SP9, and ZNF781, (b) determining if the at least two genes are hypermethylated, (c) diagnosing the subject as having at least one malignant lung nodule and/or lung cancer when hypermethylation of the at least two genes is detected, and, (d) administering an effective amount of at least one lung cancer treatment to the subject diagnosed as having at least one malignant lung nodule.

In some embodiments, the sample is a blood sample, a sputum sample, a bronchial washing sample, a bronchial brushing sample, a urine sample, or a saliva sample. In some embodiments, the sample is a blood sample.

In some embodiments, the method further comprises, prior to the measuring, collecting a blood sample from the subject, isolating plasma from the blood sample, and extracting DNA from the isolated plasma.

In some embodiments, the lung cancer treatment is selected from surgery, chemotherapy, radiation therapy, immunotherapy, and targeted drug therapy.

In some embodiments, the methylation level is measured by (a) converting unmethylated cytosine in the DNA in step (c) to uracil while leaving methylated cytosine as cytosine, and (b) measuring the level of conversion of unmethylated cytosine to uracil.

In some embodiments, the unmethylated cytosine in the DNA is converted to uracil by bisulfite treatment or enzyme treatment.

In some embodiments, the measuring is carried out by real rime polymerase chain reaction (PCR), sequencing, or microarray.

In some embodiments, the PCR is a methylation-specific quantitative real-time PCR.

In some embodiments, the methylation-specific quantitative real-time PCR uses a forward primer comprising SEQ ID NO: 1, a reverse primer comprising SEQ ID NO: 2, and a probe comprising SEQ ID NO: 3.

In some embodiments, the methylation-specific quantitative real-time PCR uses a forward primer comprising SEQ ID NO: 4, a reverse primer comprising SEQ ID NO: 5, and a probe comprising SEQ ID NO: 6.

In some embodiments, the methylation-specific quantitative real-time PCR uses a forward primer comprising SEQ ID NO: 7, a reverse primer comprising SEQ ID NO: 8, and a probe comprising SEQ ID NO: 9.

In some embodiments, the methylation-specific quantitative real-time PCR uses a forward primer comprising SEQ ID NO: 10, a reverse primer comprising SEQ ID NO: 11, and a probe comprising SEQ ID NO: 12.

In some embodiments, the methylation-specific quantitative real-time PCR uses a forward primer comprising SEQ ID NO: 13, a reverse primer comprising SEQ ID NO: 14, and a probe comprising SEQ ID NO: 15.

In some embodiments, the methylation-specific quantitative real-time PCR uses a forward primer comprising SEQ ID NO: 16, a reverse primer comprising SEQ ID NO: 17, and a probe comprising SEQ ID NO: 18.

In some embodiments, the methylation level is measured by the methylation-specific high-resolution melting, pyrosequencing, nanopore long-read technology, or methylation-specific restriction enzyme digestion.

In some embodiments, the lung cancer is a non-small cell lung cancer or a small cell lung cancer.

In some embodiments, the subject has previously been determined to have at least one lung nodule.

In some embodiments, the subject is from a population with high risk of getting lung cancer.

In an aspect, the present disclosure provides a polynucleotide having a sequence of any one of SEQ ID NOs: 1-18.

In an aspect, the present disclosure provides a kit for conducting methylation-specific quantitative real-time PCR of CDO1 (the “CDO1 kit”), comprising a forward primer of SEQ ID NO: 1, a reverse primer of SEQ ID NO: 2, and a probe of SEQ ID NO: 3. In some embodiments, the kit further comprises bisulfite.

In an aspect, the present disclosure provides a kit for conducting methylation-specific quantitative real-time PCR of PTGER4 (the “PTGER4 kit”), comprising a forward primer of SEQ ID NO: 4, a reverse primer of SEQ ID NO: 5, and a probe of SEQ ID NO: 6. In some embodiments, the kit further comprises bisulfite.

In an aspect, the present disclosure provides a kit for conducting methylation-specific quantitative real-time PCR of HOXA9 (the “HOXA9 kit”), comprising a forward primer of SEQ ID NO: 7, a reverse primer of SEQ ID NO: 8, and a probe of SEQ ID NO: 9. In some embodiments, the kit further comprises bisulfite.

In an aspect, the present disclosure provides a kit for conducting methylation-specific quantitative real-time PCR of SHOX2 gene (hereinafter referred to as “SHOX2 kit”), comprising a forward primer of SEQ ID NO: 10, a reverse primer of SEQ ID NO: 11, and a probe of SEQ ID NO: 12. In some embodiments, the kit further comprises bisulfite.

In an aspect, the present disclosure provides a kit for conducting methylation-specific quantitative real-time PCR of SP9 gene (hereinafter referred to as “SP9 kit”), comprising a forward primer of SEQ ID NO: 13, a reverse primer of SEQ ID NO: 14, and a probe of SEQ ID NO: 15. In some embodiments, the kit further comprises bisulfite.

In an aspect, the present disclosure provides a kit for conducting methylation-specific quantitative real-time PCR of ZNF781 gene (hereinafter referred to as “ZNF781 kit”), comprising a forward primer of SEQ ID NO: 16, a reverse primer of SEQ ID NO: 17, and a probe of SEQ ID NO: 18. In some embodiments, the kit further comprises bisulfite.

In an aspect, the present disclosure provides a kit for determining whether at least one lung nodule found in a subject is malignant, comprising bisulfite, and reagents for conducting methylation-specific quantitative real-time PCR of at least two of the genes selected from the group consisting of CDO1, PTGER4, HOXA9, SHOX2, SP9, and ZNF781, wherein the reagents for conducting methylation-specific quantitative real-time PCR of CDO1 comprise a CDO1 forward primer, a CDO1 reverse primer, and a CDO1 probe, wherein the reagents for conducting methylation-specific quantitative real-time PCR of PTGER4 comprise a PTGER4 forward primer, a PTGER4 reverse primer, and a PTGER4 probe, wherein the reagents for conducting methylation-specific quantitative real-time PCR of HOXA9 comprise a HOXA9 forward primer, a HOXA9 reverse primer, and a HOXA9 probe, wherein the reagents for conducting methylation-specific quantitative real-time PCR of SHOX2 comprise a SHOX2 forward primer, a SHOX2 reverse primer, and a SHOX2 probe, wherein the reagents for conducting methylation-specific quantitative real-time PCR of SP9 comprise a SP9 forward primer, a SP9 reverse primer, and a SP9 probe, wherein the reagents for conducting methylation-specific quantitative real-time PCR of ZNF781 comprise a ZNF781 forward primer, a ZNF781 reverse primer, and a ZNF781 probe.

In some embodiments, the kit further comprises at least two of the kits selected from the CDO1 kit, PTGER4 kit, HOXA9 kit, SHOX2 kit, SP9 kit, and ZNF781 kit disclosed herein.

### BRIEF DESCRIPTION OF THE DRAWINGS

FIG. 1 is a receiver operating characteristics curve depicting the performance of discriminating malignant lung nodules from benign lung nodules using the methylation level of the CDO1 gene. Sensitivity is reported on the y-axis reports and 1-specificity is reported on the x-axis. Diagonal segments are produced by ties.

FIG. 2 is a receiver operating characteristics curve depicting the performance of discriminating malignant lung nodules from benign lung nodules using the methylation level of the PTGER4 gene. Sensitivity is reported on the y-axis reports and 1-specificity is reported on the x-axis. Diagonal segments are produced by ties.

FIG. 3 is a receiver operating characteristics curve depicting the performance of discriminating malignant lung nodules from benign lung nodules using the methylation level of the HOXA9 gene. Sensitivity is reported on the y-axis reports and 1-specificity is reported on the x-axis. Diagonal segments are produced by ties.

FIG. 4 is a receiver operating characteristics curve depicting the performance of discriminating malignant lung nodules from benign lung nodules using the methylation level of the CDO1 gene and the PTGER4 gene. Sensitivity is reported on the y-axis reports and 1-specificity is reported on the x-axis. Diagonal segments are produced by ties.

FIG. 5 is a receiver operating characteristics curve depicting the performance of discriminating malignant lung nodule from benign lung nodules using the methylation level of the CDO1 gene and the HOXA9 gene. Sensitivity is reported on the y-axis reports and 1-specificity is reported on the x-axis. Diagonal segments are produced by ties.

FIG. 6 is a receiver operating characteristics curve depicting the performance of discriminating malignant lung nodules from benign lung nodules using the methylation level of the PTGER4 gene and the HOXA9 gene. Sensitivity is reported on the y-axis reports and 1-specificity is reported on the x-axis. Diagonal segments are produced by ties.

FIG. 7 is a receiver operating characteristics curve depicting the performance of discriminating malignant lung nodules from benign lung nodule using the methylation level of the CDO1 gene, the PTGER4 gene, and the HOXA9 gene. Sensitivity is reported on the y-axis reports and 1-specificity is reported on the x-axis. Diagonal segments are produced by ties.

FIG. 8 is a receiver operating characteristics curve depicting the performance of discriminating malignant lung nodules from benign lung nodules using the methylation level of the SHOX2 gene. Sensitivity is reported on the y-axis reports and 1-specificity is reported on the x-axis. Diagonal segments are produced by ties.

FIG. 9 is a receiver operating characteristics curve depicting the performance of discriminating malignant lung nodules from benign lung nodules using the methylation level of the SP9 gene. Sensitivity is reported on the y-axis reports and 1-specificity is reported on the x-axis. Diagonal segments are produced by ties.

FIG. 10 is a receiver operating characteristics curve depicting the performance of discriminating malignant lung nodules from benign lung nodules using the methylation level of the ZNF781 gene. Sensitivity is reported on the y-axis reports and 1-specificity is reported on the x-axis. Diagonal segments are produced by ties.

FIG. 11 is a receiver operating characteristics curve depicting the performance of discriminating malignant lung nodules from benign lung nodules using the methylation level of the SHOX2 gene and the SP9 gene. Sensitivity is reported on the y-axis reports and 1-specificity is reported on the x-axis. Diagonal segments are produced by ties.

FIG. 12 is a receiver operating characteristics curve depicting the performance of discriminating malignant lung nodules from benign lung nodules using the methylation level of the SHOX2 gene and the PTGER4 gene. Sensitivity is reported on the y-axis reports and 1-specificity is reported on the x-axis. Diagonal segments are produced by ties.

FIG. 13 is a receiver operating characteristics curve depicting the performance of discriminating malignant lung nodules from benign lung nodules using the methylation level of the SHOX2 gene and the CDO1 gene. Sensitivity is reported on the y-axis reports and 1-specificity is reported on the x-axis. Diagonal segments are produced by ties.

FIG. 14 is a receiver operating characteristics curve depicting the performance of discriminating malignant lung nodules from benign lung nodules using the methylation level of the SHOX2 gene and the ZNF781 gene. Sensitivity is reported on the y-axis reports and 1-specificity is reported on the x-axis. Diagonal segments are produced by ties.

FIG. 15 is a receiver operating characteristics curve depicting the performance of discriminating malignant lung nodules from benign lung nodules using the methylation level of the SP9 gene and the PTGER4 gene. Sensitivity is reported on the y-axis reports and 1-specificity is reported on the x-axis. Diagonal segments are produced by ties.

FIG. 16 is a receiver operating characteristics curve depicting the performance of discriminating malignant lung nodules from benign lung nodules using the methylation level of the SP9 gene and the CDO1 gene. Sensitivity is reported on the y-axis reports and 1-specificity is reported on the x-axis. Diagonal segments are produced by ties.

FIG. 17 is a receiver operating characteristics curve depicting the performance of discriminating malignant lung nodules from benign lung nodules using the methylation level of the SP9 gene and the ZNF781 gene. Sensitivity is reported on the y-axis reports and 1-specificity is reported on the x-axis. Diagonal segments are produced by ties.

FIG. 18 is a receiver operating characteristics curve depicting the performance of discriminating malignant lung nodules from benign lung nodules using the methylation level of the ZNF781 gene and the PTGER4 gene. Sensitivity is reported on the y-axis reports and 1-specificity is reported on the x-axis. Diagonal segments are produced by ties.

FIG. 19 is a receiver operating characteristics curve depicting the performance of discriminating malignant lung nodules from benign lung nodules using the methylation level of the ZNF781 gene and the CDO1 gene. Sensitivity is reported on the y-axis reports and 1-specificity is reported on the x-axis. Diagonal segments are produced by ties.

FIG. 20 is a receiver operating characteristics curve depicting the performance of discriminating malignant lung nodules from benign lung nodules using the methylation level of the ZNF781 gene, the CDO1 gene, and the SHOX2 gene. Sensitivity is reported on the y-axis reports and 1-specificity is reported on the x-axis. Diagonal segments are produced by ties.

FIG. 21 is a receiver operating characteristics curve depicting the performance of discriminating malignant lung nodules from benign lung nodules using the methylation level of the SHOX2 gene, the SP9 gene, and the PTGER4 gene. Sensitivity is reported on the y-axis reports and 1-specificity is reported on the x-axis. Diagonal segments are produced by ties.

FIG. 22 is a receiver operating characteristics curve depicting the performance of discriminating malignant lung nodules from benign lung nodules using the methylation level of the SHOX2 gene, the SP9 gene, and the CDO1 gene. Sensitivity is reported on the y-axis reports and 1-specificity is reported on the x-axis. Diagonal segments are produced by ties.

FIG. 23 is a receiver operating characteristics curve depicting the performance of discriminating malignant lung nodules from benign lung nodules using the methylation level of the SHOX2 gene, the SP9 gene, and the ZNF781 gene. Sensitivity is reported on the y-axis reports and 1-specificity is reported on the x-axis. Diagonal segments are produced by ties.

FIG. 24 is a receiver operating characteristics curve depicting the performance of discriminating malignant lung nodules from benign lung nodules using the methylation level of the SHOX2 gene, the PTGER4 gene, and the CDO1 gene. Sensitivity is reported on the y-axis reports and 1-specificity is reported on the x-axis. Diagonal segments are produced by ties.

FIG. 25 is a receiver operating characteristics curve depicting the performance of discriminating malignant lung nodules from benign lung nodules using the methylation level of the SHOX2 gene, the PTGER4 gene, and the ZNF781 gene. Sensitivity is reported on the y-axis reports and 1-specificity is reported on the x-axis. Diagonal segments are produced by ties.

FIG. 26 is a receiver operating characteristics curve depicting the performance of discriminating malignant lung nodules from benign lung nodules using the methylation level of the SP9 gene, the PTGER4 gene, and the CDO1 gene. Sensitivity is reported on the y-axis reports and 1-specificity is reported on the x-axis. Diagonal segments are produced by ties.

FIG. 27 is a receiver operating characteristics curve depicting the performance of discriminating malignant lung nodules from benign lung nodules using the methylation level of the SP9 gene, the PTGER4 gene, and the ZNF781 gene. Sensitivity is reported on the y-axis reports and 1-specificity is reported on the x-axis. Diagonal segments are produced by ties.

FIG. 28 is a receiver operating characteristics curve depicting the performance of discriminating malignant lung nodules from benign lung nodules using the methylation level of the PTGER4 gene, the CDO1 gene, and the ZNF781 gene. Sensitivity is reported on the y-axis reports and 1-specificity is reported on the x-axis. Diagonal segments are produced by ties.

FIG. 29 is a receiver operating characteristics curve depicting the performance of discriminating malignant lung nodules from benign lung nodules using the methylation level of the SHOX2 gene, the SP9 gene, the PTGER4 gene, and the CDO1 gene. Sensitivity is reported on the y-axis reports and 1-specificity is reported on the x-axis. Diagonal segments are produced by ties.

FIG. 30 is a receiver operating characteristics curve depicting the performance of discriminating malignant lung nodules from benign lung nodules using the methylation level of the SHOX2 gene, the SP9 gene, the PTGER4 gene, and the ZNF781 gene. Sensitivity is reported on the y-axis reports and 1-specificity is reported on the x-axis. Diagonal segments are produced by ties.

FIG. 31 is a receiver operating characteristics curve depicting the performance of discriminating malignant lung nodules from benign lung nodules using the methylation level of the SP9 gene, the PTGER4 gene, the CDO1 gene, and the ZNF781 gene. Sensitivity is reported on the y-axis reports and 1-specificity is reported on the x-axis. Diagonal segments are produced by ties.

FIG. 32 is a receiver operating characteristics curve depicting the performance of discriminating malignant lung nodules from benign lung nodules using the methylation level of the SP9 gene, the PTGER4 gene, the CDO1 gene, the ZNF781 gene, and the SHOX2 gene. Sensitivity is reported on the y-axis reports and 1-specificity is reported on the x-axis. Diagonal segments are produced by ties.

FIG. 33 is a receiver operating characteristics curve depicting the performance of discriminating malignant lung nodules from benign lung nodules using the methylation level of the SP9 gene, the CDO1 gene, and the ZNF781 gene. Sensitivity is reported on the y-axis reports and 1-specificity is reported on the x-axis. Diagonal segments are produced by ties.

FIG. 34 is a receiver operating characteristics curve depicting the performance of discriminating malignant lung nodules from benign lung nodules using the methylation level of the SHOX2 gene, the SP9 gene, the CDO1 gene, the ZNF781 gene. Sensitivity is reported on the y-axis reports and 1-specificity is reported on the x-axis. Diagonal segments are produced by ties.

FIG. 35 is a receiver operating characteristics curve depicting the performance of discriminating malignant lung nodules from benign lung nodules using the methylation level of the SHOX2 gene, the PTGER4 gene, the CDO1 gene, and the ZNF781 gene. Sensitivity is reported on the y-axis reports and 1-specificity is reported on the x-axis. Diagonal segments are produced by ties.

FIG. 36 is the gene sequence of the CDO1 gene, wherein the CpG sites are highlighted.

FIG. 37 is the gene sequence of the PTGER4 gene, wherein the CpG sites are highlighted.

FIG. 38 is the gene sequence of the HOXA9 gene, wherein the CpG sites are highlighted.

FIG. 39 is the gene sequence of the SHOX2 gene, wherein the CpG sites are highlighted.

FIG. 40 is the gene sequence of the SP9 gene, wherein the CpG sites are highlighted.

FIG. 41 is the gene sequence of the ZNF781 gene, wherein the CpG sites are highlighted.

### DETAILED DESCRIPTION

### Definitions

In the present disclosure, unless otherwise specified, the scientific and technical terms used herein have the meanings generally understood by a person skilled in the art. Accordingly, the terms defined herein are more fully described by reference to the Specification as a whole.

As used herein, the singular terms “a,” “an,” and “the” include the plural reference unless the context clearly indicates otherwise.

As used herein, “and/or” refers to and encompasses any and all possible combinations of one or more of the associated listed items, as well as the lack of combinations when interpreted in the alternative (“or”). Moreover, the present disclosure also contemplates that in some embodiments of the disclosure, any feature or combination of features set forth herein can be excluded or omitted.

Unless the context requires otherwise, the terms “comprise,” “comprises,” and “comprising,” or similar terms are intended to mean a non-exclusive inclusion, such that a recited list of elements or features does not include those stated or listed elements solely, but may include other elements or features that are not listed or stated.

Unless otherwise indicated, nucleic acids are written left to right in the 5′ to 3′ orientation.

It is to be understood that this disclosure is not limited to the particular methodology, protocols, and reagents described, as these may vary, depending upon the context in which they are used by those of skills in the art.

As used herein, “nodule” refers to an abnormal growth or lump of cells. Lung nodules, which are also referred to as pulmonary nodules, are nodules that are formed in a lung.

As used herein, “malignant” or “malignancy” refers to the presence of cancerous cells that have the ability to spread to other sites in the body or to invade nearby tissues and destroy them.

As used herein, a “minimally invasive” procedure refers to a procedure that does not involve an incision into the body. In some embodiments, the minimally invasive procedure involves a needle puncture. In some embodiments, the minimally invasive procedure is bronchial washing or bronchial brushing. For the purpose of this application, minimally invasive methods include taking a blood sample, a sputum sample, a urine sample, a sample collected from bronchial washing, a sample collected from bronchial brushing, or a saliva sample from a subject.

As used herein, a “subject” refers to any animal including, but not limited to human, non-human primate, rodent, and the like, to which the methods of the present disclosure are administered. Typically, the term “subject” and “patient” are used interchangeably herein in reference to a human subject.

For the purpose of the present application, “methylation level” refers to the number of 5 mC bases contained within a gene. As used herein, a target gene is “hypermethylated” if the target gene extracted from a subject's sample has more 5 mC than a corresponding gene in a sample from a subject that does not have malignant lung nodule. In some embodiments where methylation level is measured by methylation-specific qPCR, a target gene is hypermethylated if ΔCt≤15.5, wherein ΔCt is calculated as candidate gene Ct—reference gene Ct. More information about methylation-specific qPCR is provided in a later section of this disclosure.

As used herein, “lung cancer” includes all types of cancer that starts in the lungs. Exemplary lung cancers include, but are not limited to, adenocarcinoma, large cell carcinoma, squamous cell carcinoma, small cell carcinoma, combined small cell carcinoma, and mesothelioma.

As used herein, “reference gene” refers to a gene that exists in all cells and is used for internal reaction control purposes so that differences in the amount and quality of starting nucleic acid and in PCR amplification can be normalized. Generally speaking, expression levels of a reference gene do not significantly vary among tissues and experimental situations analyzed (Radonic et al., 2004). In some embodiments, the reference gene is selected from the basic metabolism genes (called Housekeeping Genes—HKGs) which, by definition, being involved in processes essential for the survival of cells, are expressed in a stable and non-regulated constant level (Thellin et al. 1999).

### DNA Methylation

DNA methylation is an epigenetic mechanism that regulates gene expression and cell differentiation. In mammals, DNA methylation is essential for normal development and is associated with a number of key processes including genomic imprinting, X-chromosome inactivation, repression of transposable elements, aging, and carcinogenesis.

Natural enzymatic DNA methylation has been found to take place on two nucleobases, cytosine and adenine. The modified bases are 5-methylcytosine (5 mC), N⁴-methylcytosine (4 mC), and N⁶-methyladenine (6 mA). The latter (6 mA and 4 mC) are restricted to prokaryotes and certain eukaryotes. In mammals, 5 mC is the dominant form of DNA methylation, which mainly occurs in the context of cytosine-phosphate-guanine (CpG) dinucleotides, usually with the cytosines on both strands being methylated. The CpG sites or CG sites are regions of DNA where a cytosine nucleotide is followed by a guanine nucleotide in the linear sequence of bases in 5′ to 3′ direction. Mammalian genomes exhibit particularly high CpG methylation levels. Although there are some tissue-specific differences, about 70-80% of CpGs are methylated. While the majority of CpGs are methylated, regions of densely clustered CpGs, known as CpG islands (CGIs), are often devoid of methylation. Many CGIs are found in the vicinity of gene promoters, with approximately two-thirds of genes having a CGI at their promoter. Methylation of promoter CGIs provokes long-term transcriptional repression of the associated genes.

In mammals, 5 mC methylation is catalyzed by a family of DNA methyltransferases (Dnmts) that transfer a methyl group from S-adenyl methionine to the fifth carbon of a cytosine residue to form 5 mC. DNMT3a and DNMT3b are the de novo methyltransferases that set up DNA methylation patterns early in development. DNMT3L is a protein that is homologous to the other DNMT3s but has no catalytic activity. Instead, DNMT3L assists the de novo methyltransferases by increasing their ability to bind to DNA and stimulating their activity. DNMT1 is the proposed maintenance methyltransferase that is responsible for copying DNA methylation patterns to the daughter strands during DNA replication.

### Malignant Lung Nodule and Lung Cancer

Lung cancer is the most common cause of global cancer-related mortality, leading to over a million deaths each year. Patients who present with advanced stage lung cancer usually have poor prognosis. Malignant lung nodules usually indicate early stages of lung cancer. Therefore, distinguishing malignant lung nodules from benign lung nodules is very important for early detection of lung cancer, and for making a suitable treatment plan for the patient.

Over the last few decades, studies have been showing that alterations in DNA methylation patterns can distinguish cancer cells from normal cells. Aberrant DNA methylation results in dysregulation of various genes and occurs in all stages of lung cancer, including initiation, growth, and metastasis. It has been reported that changes in DNA methylation patterns occur in a developmental stage and tissue specific manner, and often accompany cancer development. During carcinogenesis, both alleles of a tumor suppressor gene need to be inactivated by genomic changes such as chromosomal deletions or loss-of-function mutations in the coding region of a gene. As an alternative mechanism, transcriptional silencing by hypermethylation of CpG islands spanning the promoter regions of tumor suppressor genes is a common and important process in carcinogenesis. Since hypermethylation generally leads to inactivation of gene expression, this epigenetic alteration is considered to be a key mechanism for long-term silencing of tumor suppressor genes (Esteller, 2002; Wajed et al., 2001). The importance of promoter methylation in functional inactivation of lung cancer suppressor genes is becoming increasingly recognized. It is estimated that between 0.5% and 3% of all genes carrying CpG islands may be silenced by DNA methylation in lung cancer (Costello et al., 2000).

The present disclosure provides minimally invasive methods to determine whether at least one lung nodule found in a subject is malignant by measuring DNA methylation levels of a combination of genes. Since malignant lung nodules are cancerous and can develop into lung cancer, the methods provided herein can also be used to detect lung cancer. The methods disclosed herein also provide helpful guidance to doctors in determining a proper treatment plan for a subject having at least one malignant lung nodule.

### Cell-Free Circulating DNA

Cell-free circulating DNA (cfDNA) are DNA fragments that can be isolated from mammalian blood serum or plasma. The existence of extracellular nucleic acids in the circulation was first reported by Mandel and Metais in 1948 (Mandel et al., 1948). In 1989, Stroun et al. showed that DNA circulating in cancer patients exhibits some characteristic features of tumor DNA, such as decreased strand stability (Stroun et al., 1989). In some embodiments, cfDNA extracted from a blood sample is used in the methods disclosed herein. In some embodiments, cfDNA extracted from a urine sample is used in the methods disclosed herein. In some embodiments, cfDNA extracted from a saliva sample is used in the methods disclosed herein.

The concentration of cfDNA is generally low in healthy individuals, and generally ranges from 0 to 100 ng/mL, since defective cells are efficiently removed from the circulation by phagocytes (Elshimali et al., 2013). In cancer patients, increased amounts of cfDNA are released by necrotic and apoptotic tumor cells, but the levels can vary widely with a range from 0 to >1000 ng/mL (Schwarzenbach et al., 2011, Fleischhacker et al., 2007). Since molecular alterations can be detected in cfDNA, analysis of cfDNA isolated from plasma or serum provides a minimally invasive approach for studying epigenetic changes in cancer patients. For example, high levels of TSG promoter methylation were revealed in cfDNA of lung cancer patients, demonstrating that specific epigenetic alterations, detected in the genomic DNA in tumor cells, could also be found in patient serum/plasma (Fujiwara et al., 2005, Sigalotti et al., 2019).

### Methods

In an aspect, the present disclosure provides a method comprising (a) measuring the methylation level of at least two genes in a sample from a subject, wherein the at least two genes are selected from CDO1, PTGER4, HOXA9, SHOX2, SP9, and ZNF781, and (b) determining if the at least two genes are hypermethylated, wherein detection of hypermethylation of the at least two genes indicates that the subject has at least one malignant lung nodule and/or lung cancer.

In some embodiments, the method comprises (a) collecting a sample from the subject; (b) extracting DNA from the sample; (c) measuring the methylation level of at least two genes in the extracted DNA, wherein the at least two genes are selected from CDO1 (SEQ ID NO: 19), PTGER4 (SEQ ID NO: 20), HOXA9 (SEQ ID NO: 21), SHOX2 (SEQ ID NO: 22), SP9 (SEQ ID NO: 23), and ZNF781 (SEQ ID NO: 24); and (d) determining if the at least two genes are hypermethylated, wherein detection of hypermethylation of the at least two genes indicates that the subject has at least one malignant lung nodule and/or lung cancer. In some embodiments, the sample is a blood sample, a sputum sample, a sample collected from bronchial washing, a sample collected from bronchial brushing, a urine sample, or a saliva sample. In some embodiment, the sample is a blood sample.

In some embodiments, the method comprises (a) collecting blood sample from the subject; (b) isolating plasma from the blood sample; (c) extracting DNA from the isolated plasma; (d) measuring the methylation level of at least two genes in the extracted DNA, wherein the at least two genes are selected from CDO1, PTGER4, HOXA9, SHOX2, SP9, and ZNF781; and (e) determining if the at least two genes are hypermethylated, wherein detection of hypermethylation of the at least two genes indicates that the subject has at least one malignant lung nodule and/or lung cancer.

In some embodiments, the methylation level of CDO1 and PTGER4 are measured, and detection of hypermethylation of both CDO1 and PTGER4 indicates that the subject has at least one malignant lung nodule and/or lung cancer.

In some embodiments, the methylation level of CDO1 and HOXA9 are measured, and detection of hypermethylation of both CDO1 and HOXA9 indicates that the subject has at least one malignant lung nodule and/or lung cancer.

In some embodiments, the methylation level of CDO1 and SHOX2 are measured, and detection of hypermethylation of both CDO1 and SHOX2 indicates that the subject has at least one malignant lung nodule and/or lung cancer.

In some embodiments, the methylation level of CDO1 and SP9 are measured, and detection of hypermethylation of both CDO1 and SP9 indicates that the subject has at least one malignant lung nodule and/or lung cancer.

In some embodiments, the methylation level of CDO1 and ZNF781 are measured, and detection of hypermethylation of both CDO1 and ZNF781 indicates that the subject has at least one malignant lung nodule and/or lung cancer.

In some embodiments, the methylation level of PTGER4 and HOXA9 are measured, and detection of hypermethylation of both PTGER4 and HOXA9 indicates that the subject has at least one malignant lung nodule and/or lung cancer.

In some embodiments, the methylation level of PTGER4 and SHOX2 are measured, and detection of hypermethylation of both PTGER4 and SHOX2 indicates that the subject has at least one malignant lung nodule and/or lung cancer.

In some embodiments, the methylation level of PTGER4 and SP9 are measured, and detection of hypermethylation of both PTGER4 and SP9 indicates that the subject has at least one malignant lung nodule and/or lung cancer.

In some embodiments, the methylation level of PTGER4 and ZNF781 are measured, and detection of hypermethylation of both PTGER4 and ZNF781 indicates that the subject has at least one malignant lung nodule and/or lung cancer.

In some embodiments, the methylation level of HOXA9 and SHOX2 are measured, and detection of hypermethylation of both HOXA9 and SHOX2 indicates that the subject has at least one malignant lung nodule and/or lung cancer.

In some embodiments, the methylation level of HOXA9 and SP9 are measured, and detection of hypermethylation of both HOXA9 and SP9 indicates that the subject has at least one malignant lung nodule and/or lung cancer.

In some embodiments, the methylation level of HOXA9 and ZNF781 are measured, and detection of hypermethylation of both HOXA9 and ZNF781 indicates that the subject has at least one malignant lung nodule and/or lung cancer.

In some embodiments, the methylation level of SHOX2 and SP9 are measured, and detection of hypermethylation of both SHOX2 and SP9 indicates that the subject has at least one malignant lung nodule and/or lung cancer.

In some embodiments, the methylation level of SHOX2 and ZNF781 are measured, and detection of hypermethylation of both SHOX2 and ZNF781 indicates that the subject has at least one malignant lung nodule and/or lung cancer.

In some embodiments, the methylation level of SP9 and ZNF781 are measured, and detection of hypermethylation of both SP9 and ZNF781 indicates that the subject has at least one malignant lung nodule and/or lung cancer.

In some embodiments, the methylation level of CDO1, PTGER4 and HOXA9 are measured, and detection of hypermethylation of all of CDO1, PTGER4 and HOXA9 indicates that the subject has at least one malignant lung nodule and/or lung cancer.

In some embodiments, the methylation level of CDO1, PTGER4 and SHOX2 are measured, and detection of hypermethylation of all of CDO1, PTGER4 and SHOX2 indicates that the subject has at least one malignant lung nodule and/or lung cancer.

In some embodiments, the methylation level of CDO1, PTGER4 and SP9 are measured, and detection of hypermethylation of all of CDO1, PTGER4 and SP9 indicates that the subject has at least one malignant lung nodule and/or lung cancer.

In some embodiments, the methylation level of CDO1, PTGER4 and ZNF781 are measured, and detection of hypermethylation of all of CDO1, PTGER4 and SP9 indicates that the subject has at least one malignant lung nodule and/or lung cancer.

In some embodiments, the methylation level of CDO1, HOXA9, and SHOX2 are measured, and detection of hypermethylation of all of CDO1, HOXA9, and SHOX2 indicates that the subject has at least one malignant lung nodule and/or lung cancer.

In some embodiments, the methylation level of CDO1, HOXA9, and SP9 are measured, and detection of hypermethylation of all of CDO1, HOXA9, and SP9 indicates that the subject has at least one malignant lung nodule and/or lung cancer.

In some embodiments, the methylation level of CDO1, HOXA9, and ZNF781 are measured, and detection of hypermethylation of all of CDO1, HOXA9, and ZNF781 indicates that the subject has at least one malignant lung nodule and/or lung cancer.

In some embodiments, the methylation level of CDO1, SHOX2, and SP9 are measured, and detection of hypermethylation of all of CDO1, SHOX2, and SP9 indicates that the subject has at least one malignant lung nodule and/or lung cancer.

In some embodiments, the methylation level of CDO1, SHOX2, and ZNF781 are measured, and detection of hypermethylation of all of CDO1, SHOX2, and ZNF781 indicates that the subject has at least one malignant lung nodule and/or lung cancer.

In some embodiments, the methylation level of CDO1, SP9, and ZNF781 are measured, and detection of hypermethylation of all of CDO1, SP9, and ZNF781 indicates that the subject has at least one malignant lung nodule and/or lung cancer.

In some embodiments, the methylation level of PTGER4, HOXA9, and SHOX2 are measured, and detection of hypermethylation of all of PTGER4, HOXA9, and SHOX2 indicates that the subject has at least one malignant lung nodule and/or lung cancer.

In some embodiments, the methylation level of PTGER4, HOXA9, and SP9 are measured, and detection of hypermethylation of all of PTGER4, HOXA9, and SP9 indicates that the subject has at least one malignant lung nodule and/or lung cancer.

In some embodiments, the methylation level of PTGER4, HOXA9, and ZNF781 are measured, and detection of hypermethylation of all of PTGER4, HOXA9, and ZNF781 indicates that the subject has at least one malignant lung nodule and/or lung cancer.

In some embodiments, the methylation level of PTGER4, SHOX2, and SP9 are measured, and detection of hypermethylation of all of PTGER4, SHOX2 and SP9 indicates that the subject has at least one malignant lung nodule and/or lung cancer.

In some embodiments, the methylation level of PTGER4, SHOX2, and ZNF781 are measured, and detection of hypermethylation of all of PTGER4, SHOX2, and ZNF781 indicates that the subject has at least one malignant lung nodule and/or lung cancer.

In some embodiments, the methylation level of PTGER4, SP9, and ZNF781 are measured, and detection of hypermethylation of all of PTGER4, SP9, and ZNF781 indicates that the subject has at least one malignant lung nodule and/or lung cancer.

In some embodiments, the methylation level of HOXA9, SHOX2, and SP9 are measured, and detection of hypermethylation of all of HOXA9, SHOX2, and SP9 indicates that the subject has at least one malignant lung nodule and/or lung cancer.

In some embodiments, the methylation level of HOXA9, SHOX2, and ZNF781 are measured, and detection of hypermethylation of all of HOXA9, SHOX2, and ZNF781 indicates that the subject has at least one malignant lung nodule and/or lung cancer.

In some embodiments, the methylation level of HOXA9, SP9, and ZNF781 are measured, and detection of hypermethylation of all of HOXA9, SP9, and ZNF781 indicates that the subject has at least one malignant lung nodule and/or lung cancer.

In some embodiments, the methylation level of SHOX2, SP9, and ZNF781 are measured, and detection of hypermethylation of all of SHOX2, SP9, and ZNF781 indicates that the subject has at least one malignant lung nodule and/or lung cancer.

In some embodiments, the methylation level of CDO1, PTGER4, HOXA9, and SHOX2 are measured, and detection of hypermethylation of all of CDO1, PTGER4, HOXA9, and SHOX2 indicates that the subject has at least one malignant lung nodule and/or lung cancer.

In some embodiments, the methylation level of CDO1, PTGER4, HOXA9, and SP9 are measured, and detection of hypermethylation of all of CDO1, PTGER4, HOXA9, and SP9 indicates that the subject has at least one malignant lung nodule and/or lung cancer.

In some embodiments, the methylation level of CDO1, PTGER4, HOXA9, and ZNF781 are measured, and detection of hypermethylation of all of CDO1, PTGER4, HOXA9, and ZNF781 indicates that the subject has at least one malignant lung nodule and/or lung cancer.

In some embodiments, the methylation level of CDO1, PTGER4, SHOX2, and SP9 are measured, and detection of hypermethylation of all of CDO1, PTGER4, SHOX2, and SP9 indicates that the subject has at least one malignant lung nodule and/or lung cancer.

In some embodiments, the methylation level of CDO1, PTGER4, SHOX2, and ZNF781 are measured, and detection of hypermethylation of all of CDO1, PTGER4, SHOX2, and ZNF781 indicates that the subject has at least one malignant lung nodule and/or lung cancer.

In some embodiments, the methylation level of CDO1, PTGER4, SP9, and ZNF781 are measured, and detection of hypermethylation of all of CDO1, PTGER4, SP9, and ZNF781 indicates that the subject has at least one malignant lung nodule and/or lung cancer.

In some embodiments, the methylation level of CDO1, HOXA9, SHOX2, and SP9 are measured, and detection of hypermethylation of all of CDO1, HOXA9, SHOX2, and SP9 indicates that the subject has at least one malignant lung nodule and/or lung cancer.

In some embodiments, the methylation level of CDO1, HOXA9, SHOX2, and ZNF781 are measured, and detection of hypermethylation of all of CDO1, HOXA9, SHOX2, and ZNF781 indicates that the subject has at least one malignant lung nodule and/or lung cancer.

In some embodiments, the methylation level of CDO1, HOXA9, SP9, and ZNF781 are measured, and detection of hypermethylation of all of CDO1, HOXA9, SP9, and ZNF781 indicates that the subject has at least one malignant lung nodule and/or lung cancer.

In some embodiments, the methylation level of CDO1, SHOX2, SP9, and ZNF781 are measured, and detection of hypermethylation of all of CDO1, SHOX2, SP9, and ZNF781 indicates that the subject has at least one malignant lung nodule and/or lung cancer.

In some embodiments, the methylation level of PTGER4, HOXA9, SHOX2, and SP9 are measured, and detection of hypermethylation of all of PTGER4, HOXA9, SHOX2, and SP9 indicates that the subject has at least one malignant lung nodule and/or lung cancer.

In some embodiments, the methylation level of PTGER4, HOXA9, SHOX2, and ZNF781 are measured, and detection of hypermethylation of all of PTGER4, HOXA9, SHOX2, and ZNF781 indicates that the subject has at least one malignant lung nodule and/or lung cancer.

In some embodiments, the methylation level of PTGER4, HOXA9, SP9, and ZNF781 are measured, and detection of hypermethylation of all of PTGER4, HOXA9, SP9, and ZNF781 indicates that the subject has at least one malignant lung nodule and/or lung cancer.

In some embodiments, the methylation level of PTGER4, SHOX2, SP9, and ZNF781 are measured, and detection of hypermethylation of all of PTGER4, SHOX2, SP9, and ZNF781 indicates that the subject has at least one malignant lung nodule and/or lung cancer.

In some embodiments, the methylation level of HOXA9, SHOX2, SP9, and ZNF781 are measured, and detection of hypermethylation of all of HOXA9, SHOX2, SP9, and ZNF781 indicates that the subject has at least one malignant lung nodule and/or lung cancer.

In some embodiments, the methylation level of CDO1, PTGER4, HOXA9, SHOX2, and SP9 are measured, and detection of hypermethylation of all of CDO1, PTGER4, HOXA9, SHOX2, and SP9 indicates that the subject has at least one malignant lung nodule and/or lung cancer.

In some embodiments, the methylation level of CDO1, PTGER4, HOXA9, SHOX2, and ZNF781 are measured, and detection of hypermethylation of all of CDO1, PTGER4, HOXA9, SHOX2, and ZNF781 indicates that the subject has at least one malignant lung nodule and/or lung cancer.

In some embodiments, the methylation level of CDO1, PTGER4, HOXA9, ZNF781, and SP9 are measured, and detection of hypermethylation of all of CDO1, PTGER4, HOXA9, ZNF781, and SP9 indicates that the subject has at least one malignant lung nodule and/or lung cancer.

In some embodiments, the methylation level of CDO1, PTGER4, ZNF781, SHOX2, and SP9 are measured, and detection of hypermethylation of all of CDO1, PTGER4, ZNF781, SHOX2, and SP9 indicates that the subject has at least one malignant lung nodule and/or lung cancer.

In some embodiments, the methylation level of CDO1, ZNF781, HOXA9, SHOX2, and SP9 are measured, and detection of hypermethylation of all of CDO1, ZNF781, HOXA9, SHOX2, and SP9 indicates that the subject has at least one malignant lung nodule and/or lung cancer.

In some embodiments, the methylation level of ZNF781, PTGER4, HOXA9, SHOX2, and SP9 are measured, and detection of hypermethylation of all of ZNF781, PTGER4, HOXA9, SHOX2, and SP9 indicates that the subject has at least one malignant lung nodule and/or lung cancer.

In some embodiments, the methylation level of CDO1, PTGER4, HOXA9. SHOX2. SP9, and ZNF781 are measured, and detection of hypermethylation of all of CDO1, PTGER4, HOXA9, SHOX2. SP9, and ZNF781 indicates that the subject has at least one malignant lung nodule and/or lung cancer.

In some embodiments, the sample is a blood sample, a sputum sample, a sample collected from bronchial washing, a sample collected from bronchial brushing, a urine sample, or a saliva sample. In some embodiments, the sample is a blood sample.

Blood samples can be collected with any suitable method known in the art. Exemplary methods for collecting a blood sample include, but are not limited to, venipuncture sampling, arterial sampling, and fingerstick sampling. It would be understood that a person skilled in the art is able to determine the suitable conditions and the amount of blood to be collected for the purpose of the blood test disclosed herein.

Plasma can be collected with any suitable method known in the art. An exemplary method for collecting blood plasma is blood centrifugation. Blood separation centrifuges work by spinning blood samples (in collection tubes) at high speeds. The high rotation speeds exert a rotational force on the blood collection tubes that is referred to as a centrifugal force. When blood collection tubes are spun in a blood separation centrifuge, the centrifugal force separates the various components of blood as a function of their density and quantity in the sample. Thus, the various components of blood can be separated into different layers for easy separation. In some embodiments, one or more anticoagulants (e.g., EDTA, citrate dextrose) are added to the blood sample before centrifugation. It would be understood that a person skilled in the art is able to select conditions suitable for separating plasma from a whole blood sample. DNA can be extracted from the plasma with any suitable method known in the art. There are multiple commercially available kits for extracting DNA from plasma, for example, but not limited to, QIAamp Circulating Nucleic Acid Kit, QIAamp DNA Blood kit, and chemagic cfDNA 2k Kit H24. It would be understood that a person skilled in the art is able to select methods and conditions suitable for extracting DNA from plasma.

Sputum samples can be collected with any suitable method known in the art. Exemplary methods for collecting a sputum sample include, but are not limited to spontaneous sputum sampling, sputum induction, bronchoscopy, and gastric washing. In a spontaneous sputum sampling, a subject coughs up sputum into a sterile container. In a sputum induction, a subject inhales a saline mist which can cause a deep cough, and then coughs up sputum into a sterile container. In a bronchoscopy, a bronchoscope is passed through the mouth or nose of the subject directly into the lung, and sputum or lung tissue is removed. In a gastric washing, a tube is inserted through the subject's mouth or nose and passed into the stomach to get a sample of the gastric secretions that contain sputum that has been coughed into the throat and then swallowed. DNA can be extracted from the sputum sample with any suitable method known in the art. There are multiple commercially available kits for extracting DNA from sputum, for example, but not limited to, the Sputum DNA Isolation Kit (Cat. 46200) from Norgen Bioteck and DNeasy blood and tissue kits from Qiagen. It would be understood that a person skilled in the art is able to select methods and conditions suitable for extracting DNA from sputum.

Bronchial washing is a procedure in which cells are taken from the inside of the airways that lead to the lungs. A bronchoscope is inserted through the nose or mouth into the lungs. A mild salt solution is washed over the surface of the airways to collect cells, which are then looked at under a microscope. It would be understood that a person skilled in the art is able to select methods and conditions suitable for collecting fluid and/or cell samples from bronchial washing.

Bronchial brushing is a procedure in which a bronchoscope is inserted through the nose or mouth into the lungs. A small brush is then used to remove cells from the airways. These cells are then looked at under a microscope. Bronchial brushing is also called bronchial brush biopsy. It would be understood that a person skilled in the art is able to select methods and conditions suitable for collecting fluid and/or cell samples from bronchial brushing.

DNA can be extracted from the sample collected from bronchial washing or bronchial brushing with any suitable method known in the art. There are multiple commercially available kits for extracting DNA from samples collected from bronchial washing or bronchial brushing, for example, but not limited to, the Nucleospin Tissue Kit from Takara, and DNeasy blood and tissue kits. It would be understood that a person skilled in the art is able to select methods and conditions suitable for extracting DNA from samples collected from bronchial washing or bronchial brushing.

Urine samples can be collected with any suitable method known in the art. Exemplary methods for collecting urine sample include, but not limited to, sterile urine bag, urethral catheterization (CATH), suprapubic aspiration (SPA), or clean-catch (CC). It would be understood that a person skilled in the art is able to select methods and conditions suitable for collecting urine sample. DNA can be extracted from the urine sample with any suitable method known in the art. There are multiple commercially available kits for extracting DNA from urine, for example, but not limited to, the Quick-DNA Urine Kit from Zymo Research, the DNA Isolation Kit—Urine (ab156899) from Abcam, Urine DNA isolation kit from Norgen Biotek. It would be understood that a person skilled in the art is able to select methods and conditions suitable for extracting DNA from urine.

Saliva samples can be collected with any suitable method known in the art. In some embodiments, a saliva sample is collected using the passive drool method or the oral swab method. It would be understood that a person skilled in the art is able to select methods and conditions suitable for collecting saliva samples. DNA can be extracted from the saliva sample with any suitable method known in the art. There are multiple commercially available kits for extracting DNA from saliva, for example, but not limited to, MagMAX Saliva gDNA Isolation Kit from Thermo Fisher, the Oragene® DNA Saliva Kit, and the Saliva DNA Isolation Kit (Cat. RU45400) from Norgen Biotek. It would be understood that a person skilled in the art is able to select methods and conditions suitable for extracting DNA from saliva.

Gene methylation levels can be measured by any suitable method known in the art and disclosed herein. Methods for measuring gene methylation levels will be discussed in detail in a later section.

In an aspect, the present disclosure provides a method comprising (a) measuring the methylation level of at least two genes in a blood sample from a subject, wherein the at least two genes are selected from CDO1, PTGER4, HOXA9, SHOX2, SP9, and ZNF781, (b) determining if the at least two genes are hypermethylated, (c) diagnosing the subject as having at least one malignant lung nodule and/or lung cancer when hypermethylation of the at least two genes is detected, and, (d) administering an effective amount of at least one lung cancer treatment to the subject diagnosed as having at least one malignant lung nodule.

In some embodiments, the method comprises (a) collecting a sample from the subject; (b) extracting DNA from the sample; (c) measuring the methylation level of at least two genes in the extracted DNA, wherein the at least two genes are selected from CDO1, PTGER4, HOXA9, SHOX2, SP9, and ZNF781; and (d) determining if the at least two genes are hypermethylated, (e) diagnosing the subject as having at least one malignant lung nodule and/or lung cancer when hypermethylation of the at least two genes is detected, and, (d) administering an effective amount of at least one lung cancer treatment to the subject diagnosed as having at least one malignant lung nodule.

In some embodiments, the sample is a blood sample, a sputum sample, a sample collected from bronchial washing, a sample collected from bronchial brushing, a urine sample, or a saliva sample. In some embodiments, the sample is a blood sample.

In some embodiments, the method comprises (a) collecting blood sample from a subject; (b) isolating plasma from the blood sample; (c) extracting DNA from the isolated plasma; (d) measuring the methylation level of at least two genes in the extracted DNA, wherein the at least two genes are selected from CDO1, PTGER4, HOXA9, SHOX2, SP9, and ZNF781; (e) determining if the at least two genes are hypermethylated; (f) diagnosing the subject as having at least one malignant lung nodule and/or lung cancer when hypermethylation of the at least two genes is detected; and (g) administering an effective amount of at least one lung cancer treatment to the subject diagnosed as having at least one malignant lung nodule.

In some embodiments, the methylation level of CDO1 and PTGER4 are measured, and detection of hypermethylation of both CDO1 and PTGER4 indicates that the subject has at least one malignant lung nodule and/or lung cancer.

In some embodiments, the methylation level of CDO1 and HOXA9 are measured, and detection of hypermethylation of both CDO1 and HOXA9 indicates that the subject has at least one malignant lung nodule and/or lung cancer.

In some embodiments, the methylation level of CDO1 and SHOX2 are measured, and detection of hypermethylation of both CDO1 and SHOX2 indicates that the subject has at least one malignant lung nodule and/or lung cancer.

In some embodiments, the methylation level of CDO1 and SP9 are measured, and detection of hypermethylation of both CDO1 and SP9 indicates that the subject has at least one malignant lung nodule and/or lung cancer.

In some embodiments, the methylation level of CDO1 and ZNF781 are measured, and detection of hypermethylation of both CDO1 and ZNF781 indicates that the subject has at least one malignant lung nodule and/or lung cancer.

In some embodiments, the methylation level of PTGER4 and HOXA9 are measured, and detection of hypermethylation of both PTGER4 and HOXA9 indicates that the subject has at least one malignant lung nodule and/or lung cancer.

In some embodiments, the methylation level of PTGER4 and SHOX2 are measured, and detection of hypermethylation of both PTGER4 and SHOX2 indicates that the subject has at least one malignant lung nodule and/or lung cancer.

In some embodiments, the methylation level of PTGER4 and SP9 are measured, and detection of hypermethylation of both PTGER4 and SP9 indicates that the subject has at least one malignant lung nodule and/or lung cancer.

In some embodiments, the methylation level of PTGER4 and ZNF781 are measured, and detection of hypermethylation of both PTGER4 and ZNF781 indicates that the subject has at least one malignant lung nodule and/or lung cancer.

In some embodiments, the methylation level of HOXA9 and SHOX2 are measured, and detection of hypermethylation of both HOXA9 and SHOX2 indicates that the subject has at least one malignant lung nodule and/or lung cancer.

In some embodiments, the methylation level of HOXA9 and SP9 are measured, and detection of hypermethylation of both HOXA9 and SP9 indicates that the subject has at least one malignant lung nodule and/or lung cancer.

In some embodiments, the methylation level of HOXA9 and ZNF781 are measured, and detection of hypermethylation of both HOXA9 and ZNF781 indicates that the subject has at least one malignant lung nodule and/or lung cancer.

In some embodiments, the methylation level of SHOX2 and SP9 are measured, and detection of hypermethylation of both SHOX2 and SP9 indicates that the subject has at least one malignant lung nodule and/or lung cancer.

In some embodiments, the methylation level of SHOX2 and ZNF781 are measured, and detection of hypermethylation of both SHOX2 and ZNF781 indicates that the subject has at least one malignant lung nodule and/or lung cancer.

In some embodiments, the methylation level of SP9 and ZNF781 are measured, and detection of hypermethylation of both SP9 and ZNF781 indicates that the subject has at least one malignant lung nodule and/or lung cancer.

In some embodiments, the methylation level of CDO1, PTGER4 and HOXA9 are measured, and detection of hypermethylation of all of CDO1, PTGER4 and HOXA9 indicates that the subject has at least one malignant lung nodule and/or lung cancer.

In some embodiments, the methylation level of CDO1, PTGER4 and SHOX2 are measured, and detection of hypermethylation of all of CDO1, PTGER4 and SHOX2 indicates that the subject has at least one malignant lung nodule and/or lung cancer.

In some embodiments, the methylation level of CDO1, PTGER4 and SP9 are measured, and detection of hypermethylation of all of CDO1, PTGER4 and SP9 indicates that the subject has at least one malignant lung nodule and/or lung cancer.

In some embodiments, the methylation level of CDO1, PTGER4 and ZNF781 are measured, and detection of hypermethylation of all of CDO1, PTGER4 and ZNF781 indicates that the subject has at least one malignant lung nodule and/or lung cancer.

In some embodiments, the methylation level of CDO1, HOXA9, and SHOX2 are measured, and detection of hypermethylation of all of CDO1, HOXA9, and SHOX2 indicates that the subject has at least one malignant lung nodule and/or lung cancer.

In some embodiments, the methylation level of CDO1, HOXA9, and SP9 are measured, and detection of hypermethylation of all of CDO1, HOXA9, and SP9 indicates that the subject has at least one malignant lung nodule and/or lung cancer.

In some embodiments, the methylation level of CDO1, HOXA9, and ZNF781 are measured, and detection of hypermethylation of all of CDO1, HOXA9, and ZNF781 indicates that the subject has at least one malignant lung nodule and/or lung cancer.

In some embodiments, the methylation level of CDO1, SHOX2, and SP9 are measured, and detection of hypermethylation of all of CDO1, SHOX2, and SP9 indicates that the subject has at least one malignant lung nodule and/or lung cancer.

In some embodiments, the methylation level of CDO1, SHOX2, and ZNF781 are measured, and detection of hypermethylation of all of CDO1, SHOX2, and ZNF781 indicates that the subject has at least one malignant lung nodule and/or lung cancer.

In some embodiments, the methylation level of CDO1, SP9, and ZNF781 are measured, and detection of hypermethylation of all of CDO1, SP9, and ZNF781 indicates that the subject has at least one malignant lung nodule and/or lung cancer.

In some embodiments, the methylation level of PTGER4, HOXA9, and SHOX2 are measured, and detection of hypermethylation of all of PTGER4, HOXA9, and SHOX2 indicates that the subject has at least one malignant lung nodule and/or lung cancer.

In some embodiments, the methylation level of PTGER4, HOXA9, and SP9 are measured, and detection of hypermethylation of all of PTGER4, HOXA9, and SP9 indicates that the subject has at least one malignant lung nodule and/or lung cancer.

In some embodiments, the methylation level of PTGER4, HOXA9, and ZNF781 are measured, and detection of hypermethylation of all of PTGER4, HOXA9, and ZNF781 indicates that the subject has at least one malignant lung nodule and/or lung cancer.

In some embodiments, the methylation level of PTGER4, SHOX2, and SP9 are measured, and detection of hypermethylation of all of PTGER4, SHOX2, and SP9 indicates that the subject has at least one malignant lung nodule and/or lung cancer.

In some embodiments, the methylation level of PTGER4, SHOX2, and ZNF781 are measured, and detection of hypermethylation of all of PTGER4, SHOX2, and ZNF781 indicates that the subject has at least one malignant lung nodule and/or lung cancer.

In some embodiments, the methylation level of PTGER4, SP9, and ZNF781 are measured, and detection of hypermethylation of all of PTGER4, SP9, and ZNF781 indicates that the subject has at least one malignant lung nodule and/or lung cancer.

In some embodiments, the methylation level of HOXA9, SHOX2, and SP9 are measured, and detection of hypermethylation of all of HOXA9, SHOX2, and SP9 indicates that the subject has at least one malignant lung nodule and/or lung cancer.

In some embodiments, the methylation level of HOXA9, SHOX2, and ZNF781 are measured, and detection of hypermethylation of all of HOXA9, SHOX2, and ZNF781 indicates that the subject has at least one malignant lung nodule and/or lung cancer.

In some embodiments, the methylation level of HOXA9, SP9, and ZNF781 are measured, and detection of hypermethylation of all of HOXA9, SP9, and ZNF781 indicates that the subject has at least one malignant lung nodule and/or lung cancer.

In some embodiments, the methylation level of SHOX2, SP9, and ZNF781 are measured, and detection of hypermethylation of all of SHOX2, SP9, and ZNF781 indicates that the subject has at least one malignant lung nodule and/or lung cancer.

In some embodiments, the methylation level of CDO1, PTGER4, HOXA9, and SHOX2 are measured, and detection of hypermethylation of all of CDO1, PTGER4, HOXA9, and SHOX2 indicates that the subject has at least one malignant lung nodule and/or lung cancer.

In some embodiments, the methylation level of CDO1, PTGER4, HOXA9, and SP9 are measured, and detection of hypermethylation of all of CDO1, PTGER4, HOXA9, and SP9 indicates that the subject has at least one malignant lung nodule and/or lung cancer.

In some embodiments, the methylation level of CDO1, PTGER4, HOXA9, and ZNF781 are measured, and detection of hypermethylation of all of CDO1, PTGER4, HOXA9, and ZNF781 indicates that the subject has at least one malignant lung nodule and/or lung cancer.

In some embodiments, the methylation level of CDO1, PTGER4, SHOX2, and SP9 are measured, and detection of hypermethylation of all of CDO1, PTGER4, SHOX2, and SP9 indicates that the subject has at least one malignant lung nodule and/or lung cancer.

In some embodiments, the methylation level of CDO1, PTGER4, SHOX2, and ZNF781 are measured, and detection of hypermethylation of all of CDO1, PTGER4, SHOX2, and ZNF781 indicates that the subject has at least one malignant lung nodule and/or lung cancer.

In some embodiments, the methylation level of CDO1, PTGER4, SP9, and ZNF781 are measured, and detection of hypermethylation of all of CDO1, PTGER4, SP9, and ZNF781 indicates that the subject has at least one malignant lung nodule and/or lung cancer.

In some embodiments, the methylation level of CDO1, HOXA9, SHOX2, and SP9 are measured, and detection of hypermethylation of all of CDO1, HOXA9, SHOX2, and SP9 indicates that the subject has at least one malignant lung nodule and/or lung cancer.

In some embodiments, the methylation level of CDO1, HOXA9, SHOX2, and ZNF781 are measured, and detection of hypermethylation of all of CDO1, HOXA9, SHOX2, and ZNF781 indicates that the subject has at least one malignant lung nodule and/or lung cancer.

In some embodiments, the methylation level of CDO1, HOXA9, SP9, and ZNF781 are measured, and detection of hypermethylation of all of CDO1, HOXA9, SP9, and ZNF781 indicates that the subject has at least one malignant lung nodule and/or lung cancer.

In some embodiments, the methylation level of CDO1, SHOX2, SP9, and ZNF781 are measured, and detection of hypermethylation of all of CDO1, SHOX2, SP9, and ZNF781 indicates that the subject has at least one malignant lung nodule and/or lung cancer.

In some embodiments, the methylation level of PTGER4, HOXA9, SHOX2, and SP9 are measured, and detection of hypermethylation of all of PTGER4, HOXA9, SHOX2, and SP9 indicates that the subject has at least one malignant lung nodule and/or lung cancer.

In some embodiments, the methylation level of PTGER4, HOXA9, SHOX2, and ZNF781 are measured, and detection of hypermethylation of all of PTGER4, HOXA9, SHOX2, and ZNF781 indicates that the subject has at least one malignant lung nodule and/or lung cancer.

In some embodiments, the methylation level of PTGER4, HOXA9, SP9, and ZNF781 are measured, and detection of hypermethylation of all of PTGER4, HOXA9, SP9, and ZNF781 indicates that the subject has at least one malignant lung nodule and/or lung cancer.

In some embodiments, the methylation level of PTGER4, SHOX2, SP9, and ZNF781 are measured, and detection of hypermethylation of all of PTGER4, SHOX2, SP9, and ZNF781 indicates that the subject has at least one malignant lung nodule and/or lung cancer.

In some embodiments, the methylation level of HOXA9, SHOX2, SP9, and ZNF781 are measured, and detection of hypermethylation of all of HOXA9, SHOX2, SP9, and ZNF781 indicates that the subject has at least one malignant lung nodule and/or lung cancer.

In some embodiments, the methylation level of CDO1, PTGER4, HOXA9, SHOX2, and SP9 are measured, and detection of hypermethylation of all of CDO1, PTGER4, HOXA9, SHOX2, and SP9 indicates that the subject has at least one malignant lung nodule and/or lung cancer.

In some embodiments, the methylation level of CDO1, PTGER4, HOXA9, SHOX2, and ZNF781 are measured, and detection of hypermethylation of all of CDO1, PTGER4, HOXA9, SHOX2, and ZNF781 indicates that the subject has at least one malignant lung nodule and/or lung cancer.

In some embodiments, the methylation level of CDO1, PTGER4, HOXA9, ZNF781, and SP9 are measured, and detection of hypermethylation of all of CDO1, PTGER4, HOXA9, ZNF781, and SP9 indicates that the subject has at least one malignant lung nodule and/or lung cancer.

In some embodiments, the methylation level of CDO1, PTGER4, ZNF781, SHOX2, and SP9 are measured, and detection of hypermethylation of all of CDO1, PTGER4, ZNF781, SHOX2, and SP9 indicates that the subject has at least one malignant lung nodule and/or lung cancer.

In some embodiments, the methylation level of CDO1, ZNF781, HOXA9, SHOX2, and SP9 are measured, and detection of hypermethylation of all of CDO1, ZNF781, HOXA9, SHOX2, and SP9 indicates that the subject has at least one malignant lung nodule and/or lung cancer.

In some embodiments, the methylation level of ZNF781, PTGER4, HOXA9, SHOX2, and SP9 are measured, and detection of hypermethylation of all of ZNF781, PTGER4, HOXA9, SHOX2, and SP9 indicates that the subject has at least one malignant lung nodule and/or lung cancer.

In some embodiments, the methylation level of CDO1, PTGER4, HOXA9, SHOX2, SP9, and ZNF781 are measured, and detection of hypermethylation of all of CDO1, PTGER4, HOXA9, SHOX2, SP9, and ZNF781 indicates that the subject has at least one malignant lung nodule and/or lung cancer.

In some embodiments, the sample is a blood sample, a sputum sample, a sample collected from bronchial washing, a sample collected from bronchial brushing, a urine sample, or a saliva sample. In some embodiments, the sample is a blood sample.

In some embodiments, the lung cancer treatment is any treatment know in the art or disclosed herein. In some embodiments, the lung cancer treatment is selected from surgery, chemotherapy, radiation therapy, immunotherapy, and targeted drug therapy.

Surgeries for lung cancer treatment include, but are not limited to, curative surgery, which removes the cancerous tumor or growth from the body; preventive surgery, which removes tissue that does not contain cancerous cells, but may develop into a malignant nodule or tumor; diagnostic surgery, which removes a tissue sample for testing and evaluation to help confirming a diagnosis; staging surgery, which works to uncover the extent of cancer, or the extent of the disease in the body; debulking surgery, which removes a portion, though not all, of a cancerous tumor or growth: palliative surgery, which is used to relieve discomfort or to correct other problems created by other cancer treatments; supportive surgery, which is used in combination with other cancer treatments to help them work effectively; and restorative surgery, which is used to restore a subject's appearance or the function of a body part after other cancer treatments or other surgeries. It would be understood that a person skilled in the art is able to select a suitable type(s) of surgery for lung cancer treatment of a subject who has been determined to carry a malignant lung nodule using the methods disclosed herein.

Chemotherapy is a type of cancer treatment that uses one or more anti-cancer drugs (chemotherapeutic agents) as part of a standardized cancer treatment regimen. Chemotherapy may be given with a curative intent or palliative intent. Chemotherapy is the application of chemicals or drugs to kill cancer cells, and its effects are systemic. There are several different classes of anticancer drugs based on their mechanisms of action, and they include the following: a) alkylating agents that damage DNA; b) anti-metabolites that replace the normal building blocks of RNA and DNA; c) antibiotics that interfere with the enzymes involved in DNA replication; d) topoisomerase inhibitors that inhibit either topoisomerase I or II, which are the enzymes involved in unwinding DNA during replication and transcription; e) mitotic inhibitors that inhibit mitosis and cell division; and f) corticosteroids, which are used for the treatment of cancer and to relieve the side effects from other drugs. Chemotherapy agents used in lung cancer treatment include, but are not limited to, xeloda, avastin, tarceva, cytoxan, taxol, taxotere, gemzar, erbitux, alimta, navelbine, platinol, trexall, ethyol, iressa, neosar, platinol-AQ, photofrin, onxol, cisplatin, taxanes, gefitinib, gemcitabine, erlotinib, amrubicin, belotecan, bendamustine, picoplatin, and palifosfarnide. It would be understood that a person skilled in the art is able to select a suitable chemotherapy regimen for lung cancer treatment of a subject who has been determined to carry a malignant lung nodule using the methods disclosed herein.

Radiation therapy is the application of high energy radiation to kill cancerous cells and to shrink a tumor or growth. At high doses, radiation therapy kills cancer cells or slows their growth by damaging their DNA. Cancer cells whose DNA is damaged beyond repair stop dividing and die. When the damaged cells die, they are broken down and removed by the body. Radiation therapy is either external or internal. External beam radiation therapy uses a machine that aims high energy rays or beams from outside the body into the tumor or growth. Types of external radiation therapy include, but are not limited to three-dimensional conformal radiation therapy (3D-CRT), image guided radiation therapy (IGRT), intensity modulated radiation therapy (IMRT), helical-tomotherapy, photon beam radiation therapy, proton beam radiation therapy, intraoperative radiation therapy (IORT), and sterotactic radiosurgery. Internal radiation therapy is a treatment in which a source of radiation is put inside the body. The radiation source can be solid or liquid. Internal radiation therapy with a solid source is called brachytherapy, in which seeds, ribbons, or capsules that contain a radiation source are placed in the subject's body, in or near the tumor or growth. Internal radiation therapy with a liquid source is called systemic therapy. Systemic means that the treatment travels in the blood to tissues throughout the body, seeking out and killing cancer cells. It would be understood that a person skilled in the art is able to select a suitable radiation therapy for lung cancer treatment of a subject who has been determined to carry a malignant lung nodule using the methods disclosed herein. Targeted drug therapy uses drugs or other substances to block the growth and spread of cancer by interfering with specific molecular targets that are involved in the growth, progression, and spread of cancer. Targeted drug therapy is also called “molecularly targeted drug therapy,” “molecularly targeted drug,” “precision medicine,” or other similar names. Many different targeted drug therapies have been approved for use in cancer treatment, including but not limited to, hormone therapies, signal transduction inhibitors, apoptosis inducers, angiogenesis inhibitors, immunotherapies, and gene therapy.

Hormone therapies slow or stop the growth of hormone-sensitive tumors or growths, which require certain hormones to grow. Hormone therapies act by preventing the body from producing the hormones or by interfering with the action of the hormones. In some embodiments, estrogen and progesterone are used to treat female patients with non-small-cell lung cancer (Katcoff et al., 2014).

Signal transduction inhibitors block the activities of molecules that participate in signal transduction, the process by which a cell responds to signals from its environment. During this process, once a cell has received a specific signal, the signal is relayed within the cell through a series of biochemical reactions that ultimately produce the appropriate response(s). In some cancers, the malignant cells are stimulated to divide continuously without being prompted to do so by external growth factors. Signal transduction inhibitors interfere with this inappropriate signaling. In some embodiments, the targeted signal pathway includes, but is not limited to, EGFR, VEGF, IGF, PI3K/Akt, TRAIL, HSP, HDAC, and EML4/ALK pathways. In some embodiments, the signal transduction pathway inhibitor used for the lung cancer treatment is BIBW2992, Vandetanib, Sunitinib, Sorafenib, Motesanib, Bevacizumab combined with erlotinib, Figitumumab, CI-994, or Crizotinib.

Apoptosis inducers cause cancer cells to undergo a process of controlled cell death called apoptosis. Apoptosis is one method the body uses to get rid of unneeded or abnormal cells, but cancer cells have strategies to avoid apoptosis. Apoptosis inducers can get around these strategies to cause the death of cancer cells. Studies have shown that cysteine-conjugated [6]-shogaol (M2), AZD6244, and Viburnum grandiflorum extract are able to induce apoptosis of lung cancer cells (Meng et al., 2010; Warin et al., 2014; Han et al., 2020).

Angiogenesis inhibitors block the growth of new blood vessels to tumors or malignant growths (a process called tumor angiogenesis). A blood supply is necessary for tumors to grow beyond a certain size because blood provides the oxygen and nutrients that tumors need for continued growth. Treatments that interfere with angiogenesis may block tumor growth. Some targeted therapies that inhibit angiogenesis interfere with the action of vascular endothelial growth factor (VEGF), a substance that stimulates new blood vessel formation. Other angiogenesis inhibitors target other molecules that stimulate new blood vessel growth. In some embodiments, the angiogenesis inhibitor used in the methods described herein is Bevacizumab or Ramucirumab.

Immunotherapies trigger the immune system to destroy cancer cells. Some immunotherapies are monoclonal antibodies that recognize specific molecules on the surface of cancer cells. Binding of the monoclonal antibody to the target molecule results in the immune system's destruction of cells that express that target molecule. Other monoclonal antibodies bind to certain immune cells to help these cells better kill cancer cells. Some monoclonal antibodies are checkpoint inhibitors. Checkpoints are proteins made by some types of immune system cells, such as T cells, and some cancer cells. These checkpoints help keep immune responses from being too strong and sometimes can keep T cells from killing cancer cells. When these checkpoints are blocked, T cells can kill cancer cells. Examples of checkpoint proteins found on T cells or cancer cells include PD-1/PD-L1 and CTLA-4/B7-1/B7-2. In some embodiments, the monoclonal antibody used in the methods described herein is Nivolumab, Pembrolizumab, or Atezolizumab (Doroshow 2019). Cancer vaccines are another type of immunotherapy as they function by boosting the immune system to fight cancer. In some embodiments, the cancer vaccine used in the methods described herein is CIMA_{VAX}-EGF.

In some embodiments the treatment involves gene therapy. Gene therapy modifies or manipulates the expression of a gene or alters the biological properties of living cells for therapeutic purposes. In some embodiments, the p53 gene therapy or TUSC2 gene therapy is used in the methods described herein.

Molecular targets for lung cancer treatment include, but are not limited to the following genes: KRAS, EGFR, ALK, ROS1, BRAF, RET, MET, and NTRK. Currently available targeted drug therapies for lung cancer include, but are not limited to, Bevacizumab, Ramucirumab, Sotorasib, Erlotinib, Afatinib, Gefitinib Osimertinib, Dacomitinib, Osimertinib, Amivantamab, Mobocertinib, Necitumumab, Crizotinib, Ceritinib, Alectinib, Brigatinib, Lorlatinib, Entrectinib, Dabrafenib, Trametinib, Selpercatinib, Pralsetinib, Capmatinib, Tempotinib, and Larotrectinib. It would be understood that a person skilled in the art is able to select a suitable targeted drug therapy for lung cancer treatment of a subject who has been determined to carry a malignant lung nodule using the methods described herein.

In some embodiments, the lung cancer treatments discussed above are used in combination. For example, in some embodiments, radiation therapy is given before surgery to shrink the size of the cancer so it can be removed by surgery and be less likely to return. In some embodiments, a combination of the chemotherapy drug topotecan and the targeted drug Berzosertib are used in combination for treating small cell lung cancer.

In some embodiments, the subject has previously been determined to have at least one lung nodule. In some embodiments, the subject has not previously been determined to have at least one lung nodule.

In some embodiments, the subject is from a general population. As used herein, a general population for the purpose of this invention refers to the entire population, including both people having or without lung nodule. In some embodiments, the subject is from a population with high risk of getting lung cancer. As used herein, a population with high risk of getting lung cancer refers to a group of people who are determined to have higher possibility of getting lung cancer. There are many reasons for a person to be determined to have higher possibility of getting lung cancer. In some embodiments, a person is determined to have a higher possibility of getting lung cancer because the person has at least one lung nodule. In some embodiments, a person is determined to have a higher possibility of getting lung cancer because the person smokes, the person has a family history of lung cancer, the person has been exposed to high level radon, or the person has received radiation therapy to the chest.

### Measuring DNA Methylation Level

Gene methylation levels can be measured by any suitable method known in the art and disclosed herein. Exemplary methods for measuring DNA methylation level are provided in Khodadadi et al., Current Advances in DNA Methylation Analysis Methods, Biomed Res Int. 2021.

In some embodiments, DNA methylation levels are measured by (1) converting unmethylated cytosine to uracil while leaving methylated cytosine as cytosine, and (2) measuring the level of conversion of unmethylated cytosine to uracil.

In some embodiments, the unmethylated cytosine is converted to uracil by bisulfite treatment. Bisulfite conversion is a three-step reaction whereby cytosines are, at low pH and high temperature, converted first to sulfonated cytosines, then deaminated to sulfonated uracils, and finally converted to uracils in a process of alkaline desulfonation. Subsequent PCR amplification converts the uracils to thymines. In the case of 5 mC, the deamination step is nearly two orders slower than for cytosine. Therefore, after bisulfite conversion, 5 mCs remain unchanged and are amplified by PCR as cytosines. Since double-stranded DNA (dsDNA) protects C from deamination, DNA typically needs to be denatured and purified prior to conversion. There are multiple commercially available kits for bisulfite conversion, including but not limited to, Bisulflash™ DNA Modification Kit. (Epigentek, P-1026), Bisulflash™ DNA Bisulfite Conversion Easy Kit (Epigentek, P-1054), Premium Bisulfite Kit (Diagenode, C02030030), Imprint® DNA Modification Kit (Sigma-Aldrich, MOD50), EZ DNA Methylation-Gold™ Kit (Zymo Research, D5005), EZ DNA Methylation-Lightning™ Kit (Zymo Research, D5030), Fast Bisulfite Conversion Kit (Abcam®, ab1127127), InnuCONVERT Bisulfite Basic kit (Analytic Jena, 845-IC-1000008), Epitect® Fast DNA Bisulfite Kit (Qiagen, 59824), Epitect® Bisulfite Kit (Qiagen, 59110), CpGenome^{T}m Turbo Bisulfite Modification Kit (Merck Millipore, 57847), and Methyleasy™ Xceed (Human Genetic Signatures, ME001) (Knit et al., 2018). Other available methods include, for example, the methods published in Wang et al., A modified bisulfite conversion method for the detection of DA methylation. Epigenomics 2017, 9, 955-969.

In some embodiments, the unmethylated cytosine in the DNA is converted to uracil by enzyme-based approaches. In some embodiments, the unmethylated cytosine in the DNA is converted to uracil using the commercially available NEBNext Enzymatic Methyl-seq kit. NEBNext Enzymatic Methyl-seq (EM-seq) is a method for identification of 5 mC and 5 hmC. The highly effective enzymatic conversion in this method minimizes damage to DNA and, with the supplied NEBNext Ultra™ II library preparation workflow reagents, produces high quality libraries that enable superior detection of 5 mC and 5 hmC from fewer sequencing reads. The gentle treatment of DNA by the steps in the EM-seq workflow minimizes damage to DNA. As a result, EM-seq converted DNA is more intact, resulting in libraries with a higher percentage of longer inserts. This enables longer sequencing reads, resulting in greater confidence in mapping, and potentially lower per-base sequencing costs depending on instrumentation and other sequencing reaction details. Additionally, the gentle treatment in the EM-seq workflow also allows maintenance of high quality DNA libraries, which can be efficiently amplified, resulting in higher library yield with fewer PCR cycles. Importantly, these higher yields are not due to the presence of PCR duplicates, which can be especially apparent at low input amounts. Indeed, EM-seq libraries display consistently low levels of duplicates across a range of input amounts. While sufficient yield of a library is desirable for successful sequencing, the quality of a library is also a factor. A high-quality library will have uniform representation of the original sample, including uniform coverage across the GC spectrum. EM-seq libraries show uniform GC coverage, highlighting the lack of damage to DNA, and that the libraries are representative of the original sample.

In some embodiments, libraries are prepared using as little as 10 ng input DNA and the supplied NEBNext Ultra 11 reagents and the optimized EM-seq Adaptor. TET2 then oxidizes 5-mC and 5-hmC, providing protection from deamination by APOBEC in the next step. In contrast, unmodified cytosines are deaminated to uracils. Libraries are then amplified using a NEBNext master mix formulation of Q5U (a modified version of Q5 High-Fidelity DNA Polymerase).

In some embodiments, the conversion products are then detected, and the level of conversion is measured, which indicates the level of methylation. In some embodiments, the measurement of methylation level after conversion is carried out by real-time polymerase chain reaction. Real-time PCR (qPCR) monitors the amplification of a targeted DNA molecule during the PCR (i.e., in real time), not at its end. Real-time PCR (qPCR) includes both quantitative PCR (quantitative real-time PCR) and semi-quantitative PCR (semi-quantitative real-time PCR). Two common methods for the detection of PCR products in real-time. PCR are (1) non-specific fluorescent dyes that intercalate with any double-stranded DNA and (2) sequence-specific DNA probes consisting of oligonucleotides that are labelled with a fluorescent reporter, which permits detection only after hybridization of the probe with its complementary sequence.

The use of non-specific fluorescent dyes involves a DNA-binding dye that binds to double-stranded (ds) DNA generated during PCR, increasing the fluorescence quantum yield of the dye. An increase in the DNA product during PCR therefore leads to an increase in fluorescence intensity measured at each cycle. In real-time PCR with dsDNA dyes the reaction is prepared as usual, with the addition of fluorescent dsDNA dye. Then the reaction is run in a real-time PCR instrument, and after each cycle, the intensity of fluorescence is measured with a detector; the dye only fluoresces when bound to the dsDNA (i.e., the PCR product). This method has the advantage of only needing a pair of primers to carry out the amplification, which keeps costs down; multiple target sequences can be monitored in a tube by using different types of dyes. However, dsDNA dyes such as SYBR Green will bind to all dsDNA PCR products, including nonspecific PCR products (such as primer dimers). This can potentially interfere with, or prevent, accurate monitoring of the intended target sequence.

Unlike non-specific fluorescent dyes, fluorescent reporter probes detect only the DNA containing the sequence complementary to the probe. Therefore, use of a reporter probe significantly increases specificity, and enables performing the technique even in the presence of other dsDNA. Using different-colored labels, fluorescent probes can be used in multiplex assays for monitoring several target sequences in the same tube. The specificity of fluorescent reporter probes also prevents interference of measurements caused by primer dimers, which are undesirable potential by-products in PCR. The method relies on a DNA-based probe with a fluorescent reporter at one end and a quencher of fluorescence at the opposite end of the probe. The close proximity of the reporter to the quencher prevents detection of its fluorescence; breakdown of the probe by the 5′ to 3′ exonuclease activity of the Taq polymerase breaks the reporter-quencher proximity and thus allows unquenched emission of fluorescence, which can be detected after excitation with a laser. An increase in the product targeted by the reporter probe at each PCR cycle therefore causes a proportional increase in fluorescence due to the breakdown of the probe and release of the reporter.

Real-time PCR can be used to quantify nucleic acids by two common methods: relative quantification and absolute quantification. Absolute quantification gives the exact number of target DNA molecules by comparison with DNA standards using a calibration curve. Relative quantification is based on internal reference genes to determine fold-differences in expression of the target gene. Relative quantification is easier to carry out than absolute quantification, as relative quantification does not require a calibration curve since the amount of the studied gene is compared to the amount of a control reference gene. The use of one or more reference genes enables the practitioner to correct for non-specific variation, such as the differences in the quantity and quality of nucleic acid used, which can affect the efficiency of reverse transcription and therefore that of the whole PCR process.

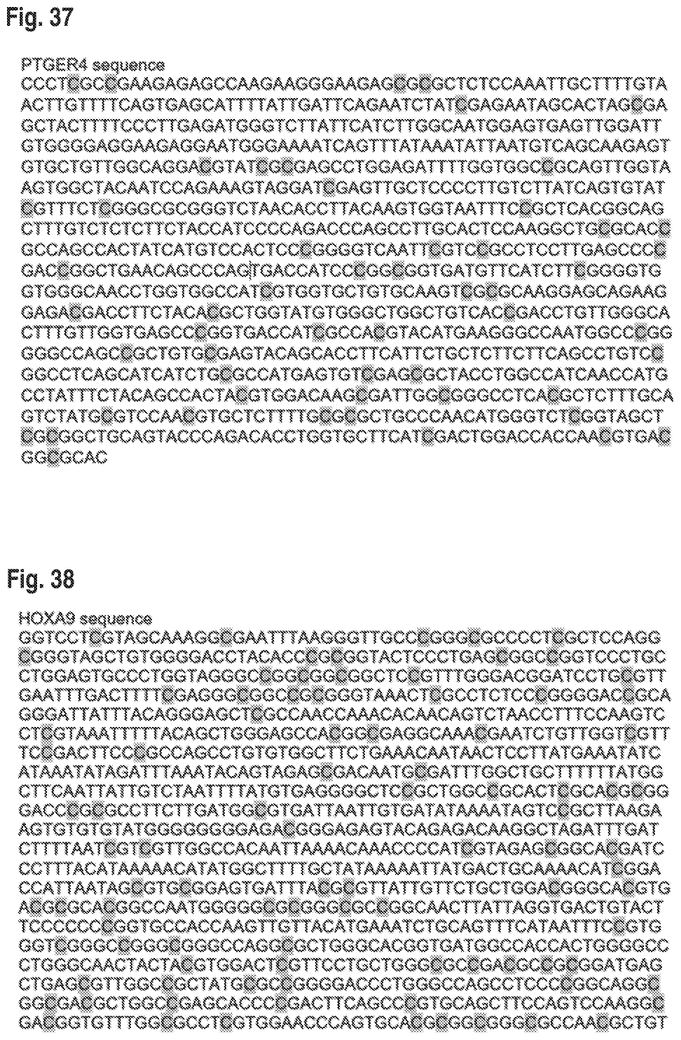

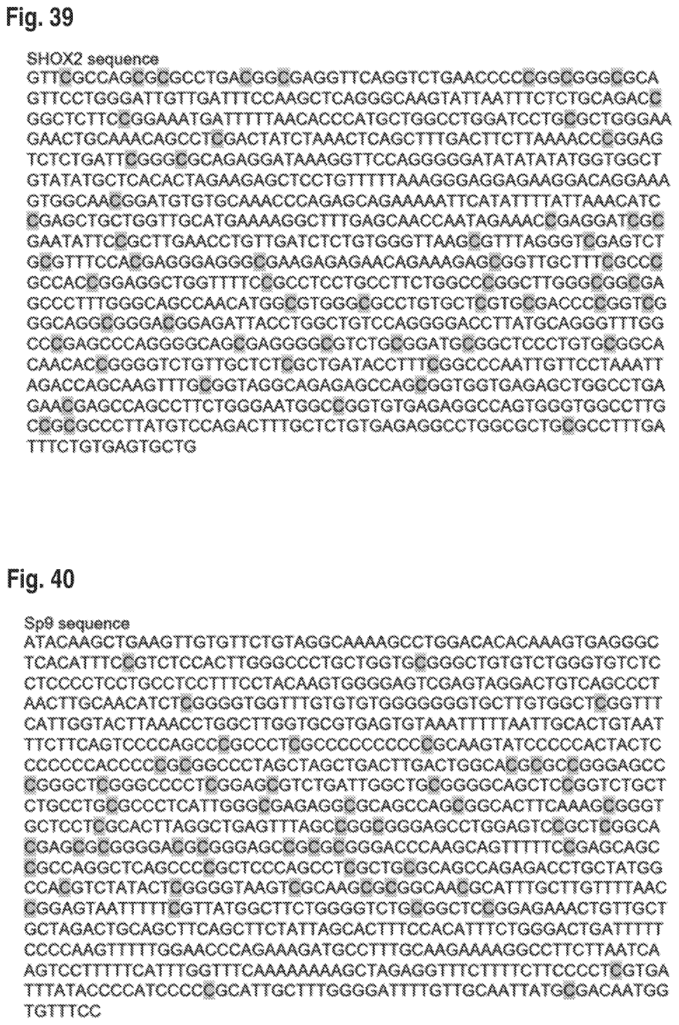

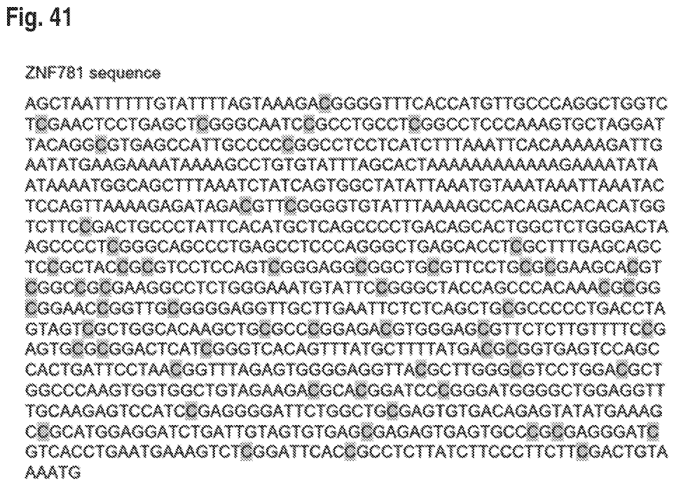

In some embodiments, the real-time PCR (qPCR) used for measuring methylation level after conversion is methylation-specific quantitative real-time PCR, which is also called methylation-specific qPCR, MS-qPCR, or qMSP. A qPCR is methylation-specific if the primer and/or probe used in the qPCR have methylation discriminatory capability. In some embodiments, “methylation discriminatory capability” is achieved by designing the primer and/or probe to bind to the converted methylated sites or converted unmethylated sites. In some embodiments, the methylation sites of interest are CpG sites. FIG. 36-41 highlights the CpG sites in CDO1, PTGER4, HOXA9, SHOX2, SP9, and ZNF781, wherein one or more of the CpG sites are methylation sites of interest.

In some embodiments, the method used for measuring methylation level after conversion is MethyLight. MethyLight is a highly sensitive assay, capable of detecting methylated alleles in the presence of a 10,000-fold excess of unmethylated alleles. In some embodiments, MethyLight comprises amplifying bisulfite-converted DNA using locus-specific PCR primers flanking an oligonucleotide probe with a 5′ fluorescent reporter dye and a 3′ quencher. In some embodiments, the 5′ fluorescent reporter dye is 6-Carboxyfluorescein (6-FAM), 5-Carboxyfluorescein (5-FAM), 5′-Dichloro-dimethoxy-fluorescein (JOE), HEX dye, VIC dye, Cy5, Cy3, TAMRA, or TET dye. In some embodiments, the 3′ quencher is Black Hole Quenchers, Iowa Black FQ, Iowa Black RQ, Dabsyl, Qxl, or the internal ZEN Quencher. The 5′ to 3′ nuclease activity of Taq DNA polymerase cleaves the probe and releases the reporter, whose fluorescence can be detected by the laser detector of, for example, the ABI Prism 7700 Sequence Detection System (Perkin-Elmer, Foster City, CA). An exemplary protocol of MethyLight is provided in Eads et al., MethyLight: a high-throughput assay to measure DA methylation, Nucleic Acids Res. 2000.

In some embodiments, a gene's methylation level is quantified using ΔCt. As used herein, “Ct” refers to the cycle threshold, which is defined as the number of cycles required for the fluorescent signal to cross the threshold (i.e., exceed background level). Ct levels are inversely proportional to the amount of target nucleic acid in the sample (i.e., the lower the Ct level, the greater the amount of target nucleic acid in the sample). Initial template quantity can be derived from its Ct. Cts<29 are considered strong positive reactions indicative of abundant target nucleic acid in the sample. Cts of 30-37 are considered positive reactions indicative of moderate amounts of target nucleic acid. Cts of 38-40 are considered weak reactions indicative of minimal amounts of target nucleic acid. A ΔCt score is calculated by subtracting a candidate gene's Ct from a reference gene's Ct. Genes most commonly applied as references in qPCR include, but are not limited to, beta actin (ACTB), glyceraldeyde-3-phosphate dehydrogenase (GAPDH), beta glucuronidase (GUSB), and hypoxanthine guanine phosphoribosyl transferase (HPRT1),

In some embodiments, the primers and probes used in the qPCR assay are designed to anneal specifically with the methylation sites of interest. In some embodiments, the probe is designed not to cover the methylation site. In some embodiments, the probe is designed to cover the methylation site, providing a greater degree of methylation discriminatory capability.

In some embodiments in which DNA methylation is measured by methylation-specific qPCR following the MethyLight protocol, a target gene is determined to be hypermethylated if ΔCt≤15.5.

In some embodiments, the methylation-specific qPCR uses a forward primer comprising SEQ ID NO: 1, a reverse primer comprising SEQ ID NO: 2, and a probe comprising SEQ ID NO: 3.

In some embodiments, the methylation-specific qPCR uses a forward primer comprising SEQ ID NO: 4, a reverse primer comprising SEQ ID NO: 5, and a probe comprising SEQ ID NO: 6.

In some embodiments, the methylation-specific qPCR uses a forward primer comprising SEQ ID NO: 7, a reverse primer comprising SEQ ID NO: 8, and a probe comprising SEQ ID NO: 9.

In some embodiments, the methylation-specific qPCR uses a forward primer comprising SEQ ID NO: 10, a reverse primer comprising SEQ ID NO: 11, and a probe comprising SEQ ID NO: 12.

In some embodiments, the methylation-specific qPCR uses a forward primer comprising SEQ ID NO: 13, a reverse primer comprising SEQ ID NO: 14, and a probe comprising SEQ ID NO: 15.

In some embodiments, the methylation-specific qPCR uses a forward primer comprising SEQ ID NO: 16, a reverse primer comprising SEQ ID NO: 17, and a probe comprising SEQ ID NO: 18.

In an aspect, the present disclosure provides a sequence of any one of SEQ ID NOs: 1-18. In some embodiments, SEQ ID NO: 1, SEQ ID NO: 2, and SEQ ID NO: 3 are used as the forward primer, the reverse primer, and the probe respectively in a methylation-specific qPCR. In some embodiments, SEQ ID NO: 4, SEQ ID NO: 5, and SEQ ID NO: 6 are used as the forward primer, the reverse primer, and the probe respectively in a methylation-specific qPCR. In some embodiments, SEQ ID NO: 7, SEQ ID NO: 8, and SEQ ID NO: 9 are used as the forward primer, the reverse primer, and the probe respectively in a methylation-specific qPCR. In some embodiments, SEQ ID NO: 10, SEQ ID NO: 11, and SEQ ID NO: 12 are used as the forward primer, the reverse primer, and the probe respectively in a methylation-specific qPCR. In some embodiments, SEQ ID NO: 13, SEQ ID NO: 14, and SEQ ID NO: 15 are used as the forward primer, the reverse primer, and the probe respectively in a methylation-specific qPCR. In some embodiments, SEQ ID NO: 16, SEQ ID NO: 17, and SEQ ID NO: 18 are used as the forward primer, the reverse primer, and the probe respectively in a methylation-specific qPCR.

In an aspect, the present disclosure provides a kit for conducting methylation-specific qPCR of CDO1 gene (hereinafter referred to as “CDO1 kit”), comprising a forward primer of SEQ ID NO: 1, a reverse primer of SEQ ID NO: 2, and a probe of SEQ ID NO: 3. In some embodiments, the kit further comprises bisulfite.

In an aspect, the present disclosure provides a kit for conducting methylation-specific qPCR of PTGER4 gene (hereinafter referred to as “PTGER4 kit”), comprising a forward primer of SEQ ID NO: 4, a reverse primer of SEQ ID NO: 5, and a probe of SEQ ID NO: 6. In some embodiments, the kit further comprises bisulfite.

In an aspect, the present disclosure provides a kit for conducting methylation-specific qPCR of HOXA9 gene (hereinafter referred to as “HOXA9 kit”), comprising a forward primer of SEQ ID NO: 7, a reverse primer of SEQ ID NO: 8, and a probe of SEQ ID NO: 9. In some embodiments, the kit further comprises bisulfite.

In an aspect, the present disclosure provides a kit for conducting methylation-specific qPCR of SHOX2 gene (hereinafter referred to as “SHOX2 kit”), comprising a forward primer of SEQ ID NO: 10, a reverse primer of SEQ ID NO: 11, and a probe of SEQ ID NO: 12 In some embodiments, the kit further comprises bisulfite.

In an aspect, the present disclosure provides a kit for conducting methylation-specific qPCR of SP9 gene (hereinafter referred to as “SP9 kit”), comprising a forward primer of SEQ ID NO: 13, a reverse primer of SEQ ID NO: 14, and a probe of SEQ ID NO: 15. In some embodiments, the kit further comprises bisulfite.

In an aspect, the present disclosure provides a kit for conducting methylation-specific qPCR of ZNF781 gene (hereinafter referred to as “ZNF781 kit”), comprising a forward primer of SEQ ID NO: 16, a reverse primer of SEQ ID NO: 17, and a probe of SEQ ID NO: 1& In some embodiments, the kit further comprises bisulfite.

In an aspect, the present disclosure provides a kit for determining whether at least one lung nodule found in a subject is malignant, comprising bisulfite, and reagents for conducting methylation-specific quantitative real-time PCR of at least two of the genes selected from the group consisting of CDO1, PTGER4, HOXA9, SHOX2, SP9, and ZNF781, wherein the reagents for conducting methylation-specific quantitative real-time PCR of CDO1 comprise a CDO1 forward primer, a CDO1 reverse primer, and a CDO1 probe, wherein the reagents for conducting methylation-specific quantitative real-time PCR of PTGER4 comprise a PTGER4 forward primer, a PTGER4 reverse primer, and a PTGER4 probe, wherein the reagents for conducting methylation-specific quantitative real-time PCR of HOXA9 comprise a HOXA9 forward primer, a HOXA9 reverse primer, and a HOXA9 probe, wherein the reagents for conducting methylation-specific quantitative real-time PCR of SHOX2 comprise a SHOX2 forward primer, a SHOX2 reverse primer, and a SHOX2 probe, wherein the reagents for conducting methylation-specific quantitative real-time PCR of SP9 comprise a SP9 forward primer, a SP9 reverse primer, and a SP9 probe, and wherein the reagents for conducting methylation-specific quantitative real-time PCR of ZNF781 comprise a ZNF781 forward primer, a ZNF781 reverse primer, and a ZNF781 probe. In some embodiments, the kit further comprises at least two of the kits selected from the CDO1 kit, the PTGER4 kit, the HOXA9 kit, the SHOX2 kit, the SP9 kit, and the ZNF781 kit as described herein.

Additionally, the sequence differences resulting from various DNA methylation patterns after the conversion could also be revealed with other methods after a methylation-nonspecific PCR amplification. In some embodiments, the method used for measuring methylation levels after conversion is methylation-specific high-resolution melting (MS-HRM). MS-HRM analysis is based on the different melting temperatures (Tm) between C-G (3H bonds) and A-T (2H bonds) pairs. Melting analysis is performed after methylation-nonspecific PCR amplification. The temperature is gradually increased and PCR products from unmethylated alleles containing A-T pairs are dissociated at a lower temperature and detected by an abrupt drop in fluorescence when an intercalating dye (e.g., SYBR Green, Eva Green, SYTO9) is released from the dsDNA. PCR amplicons of 100 bp or less are typically used for such assays to avoid the formation of secondary structures that could interfere with the analysis. Such protocols, including primer design, are explained in Wojdacz et al., Methylation-sensitive high-resolution melting, Nat Protoc. 2008. This method is relatively quick when compared with other conversion-based methods and can distinguish fully or partially methylated sites from unmethylated sites, making it particularly useful when only a small portion of analyzed sample loci is methylated. On the other hand, the method generally is not sufficiently sensitive to analyze only a single CpG. Nevertheless, this approach is semiquantitative if an unknown sample is compared with standards having a known methylated vs unmethylated ratio. Overall, MS-HRM offers a fast and relatively cost-effective estimation of methylation rate.

In some embodiments, the method used for measuring methylation level after conversion is pyrosequencing. Bisulfite pyrosequencing utilizes bisulfite conversion followed by PCR amplification with one biotinylated primer, immobilization of the amplicon on streptavidin beads, hybridization with a sequencing primer, and subsequent sequencing. This method does not give information about allele-specific methylation, but rather gives an average methylation level of both alleles in a quantitative manner. The main advantage of this technique is that it generates background free chromatograms from which the percentage of methylation is precisely calculated as C/(C+T). EpigenDx provides commercially available pyrosequencing analysis of gene-specific methylation.

It is also possible to measure DNA methylation levels without first converting unmethylated cytosine to uracil. In some embodiments, the method used for measuring methylation levels is nanopore long-read technology. DNA molecules containing methylation modifications can be sequenced in their native state without enzymatic or chemical treatment using such nanopore long-read technologies. Methylation modification can be detected by the unique signal generated by such modified bases when a DNA molecule travels through the nanopore, and can be distinguished from unmodified DNA base. More information about nanopore technologies and their use in DNA methylation measurement is provided in Simpson, J. T. et al., Detecting DNA cytosine methylation using nanopore sequencing, Nat. Methods 2017, Laszlo, A. H. et al., Detection and mapping of 5-methylcytosine and 5-hydroxymethylcytosine with nanopore MspA, Proc. Natl Acad. Sci. USA 2013, and Rand, A. C. et al., Mapping DNA methylation with high-throughput nanopore sequencing, Nat. Methods 2017.

In some embodiments, DNA methylation levels are measured by methylation-specific restriction enzyme digestion. Several site-specific methylation dependent restriction enzymes are available. For example, the endonuclease HpaI is able to digest a CCGG sequence, but only when it is un-methylated. In contrast, the MspI enzyme, which also cuts DNA at CCGG sites, is unaffected by DNA methylation. Other methylation-specific restriction enzymes include, but are not limited to HpaII, AatII, and ClaI. After digestion with one or more methylation-specific restriction enzyme(s), the location of the sites of DNA methylation is revealed by sequencing analysis, qPCR analysis using primers targeting the cutting site, or electrophoretic analysis of the digestion products. Qiagen's EpiTect Methyl II PCR Array System is a commercially available system for digestion-based DNA methylation analysis (Kurdyukove et al.; 2016, Zuo et al., 2009; Jiang et al., 2012).

### EXAMPLES

The present disclosure may be further described by the following non-limiting examples, in which standard techniques known to the skilled artisan and techniques analogous to those described in these examples may be used where appropriate. It is understood that the skilled artisan will envision additional embodiments consistent with the disclosure provided herein.

### Example 1: Whole Genome Array to Identify Candidate Genes for Malignant Lung Nodule Detection

Genomne-wide 850K Illumina methylation array analysis was performed using blood from patients with malignant and benign lung nodules. The genomic DNA was extracted from blood using QIAamp Circulating Nucleic Acid kit (Qiagen, Germany) and was treated with bisulfite conversion kit (EZ DNA Methylation-Lightning kit) from Zymo (USA) according to the manufacturer's protocol. Infinium Methylation EPIC BeadChip (850K) (Illumina, USA) was used to identify candidate differential methylation sites. The methylation status of probes was expressed as β value. CpG sites with β≥0.20 and adjusted p-value≤0.05 were defined as differential methylated sites.

### Results

The following candidates were identified from the genome-wide analysis in the clinical samples: CDO1, NEUROD1, NEUROG1, ONECUT2, PITX2, SFRP2, SLC38A4, TFAP2A, GDNF, SFRP5, CCND2, HOXA1, SIM1, GALR1, PTGER4, LOC643719, ADCY4, CIORF114, OTX1, TFAP2B, DMRTA2, TBX15, SOX1, EVX2, SOX17, SHOX2, VIPR2, TRIM58, BCL2, CDX2, TWIST1, DPP6, TBX4, CMTM2, LBXCOR, AJAP1, HOXA9, RASSF1A, MARCH11, ZNF781, RPPH1, ZIC4, TAC1, HOXA7, PRRX1, OPCML, CDKN2A, SIX3, DLX4, TMEFF2, RUNX3, GATA3, HOXA11, RARβ, PRDM14, HOXB4, HOXA10, PENK, SP9, DLEC1, MT1G, GRIN2B, CDH13, RARRES1, ITGA4, CRABP1, SEZ6L.

CDO1, PTGER4, HOXA9, SHOX2, SP9, and ZNF781 were identified as the most promising candidates to distinguish malignant lung nodules from benign nodules.

### Example 2: Detection of Malignant Lung Nodule by Methylation Level of CDO1, PTGER4, HOXA9, SHOX2, SP9, and ZNF781

Blood samples were collected from 179 lung cancer patients and 71 patients with benign lung nodules and plasma was isolated by centrifugation. DNA was extracted from the plasma samples and bisulfite-treated to convert unmethylated cytosine to uracil. The methylation status of the CDO1, PTGER4, HOXA9, SHOX2, SP9, and ZNF781 genes was measured by methylation specific quantitative real-time PCR. Primer and probe sequences used for qPCR are set forth in Table 3.

Quantitative real-time methylation-specific PCR (q-MSP) was used to detect and validate specific methylated sites. All samples were run in triplicate. Since replicates for some samples produced no detectable methylation, a Ct of 60 was used to create a near-zero value for 2^{-Δ}^{^{Ct}}, calculated for each methylation detection replicate comparing it to the mean Ct for β-Actin (ACTB). The calculation formula of mean 2^{-Δ}^{^{Ct}} value was as follows:

mean

$${2}^{{-}_{Δ} Ct = \frac{{2}^{{-}_{Δ} Ct replicate 1 + {2}^{{-}_{Δ} Ct replicate 2 + {2}^{{-}_{Δ} Ct replicate 3}}}}{3}}$$

### Results

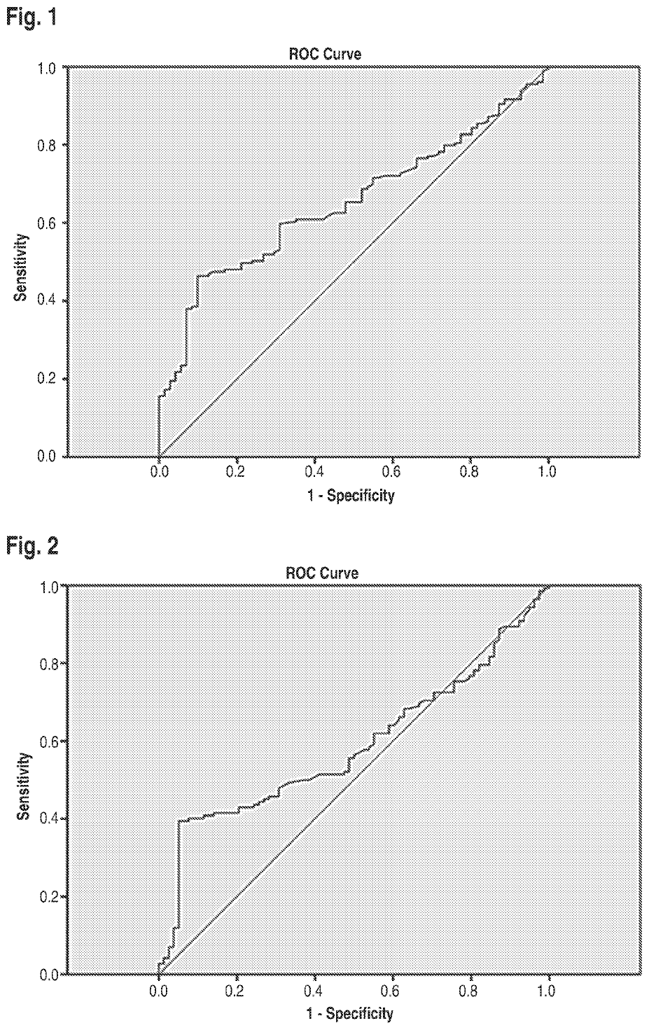

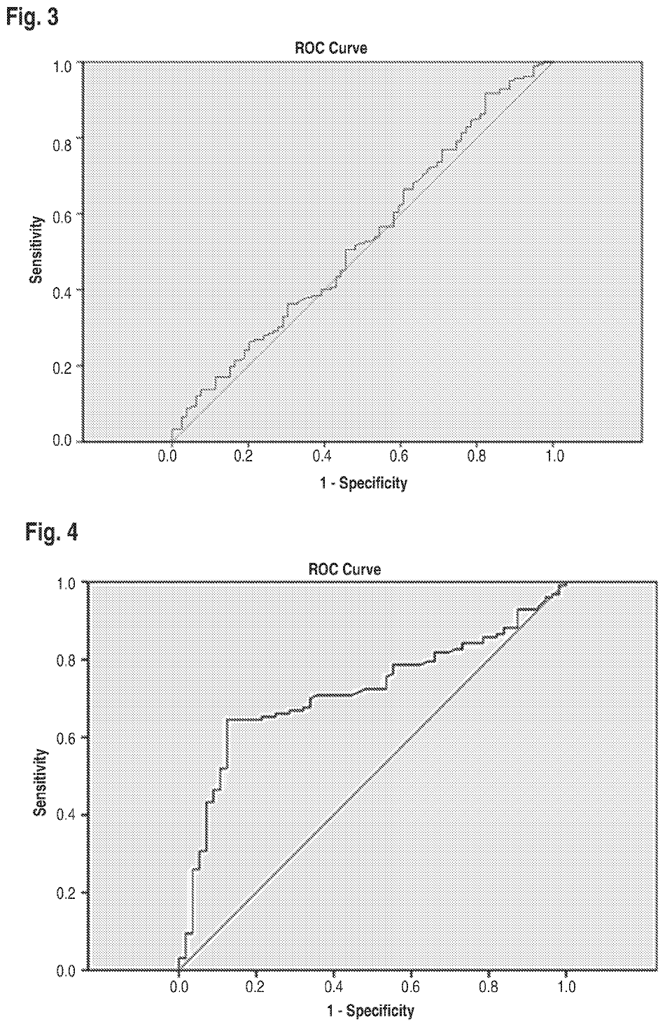

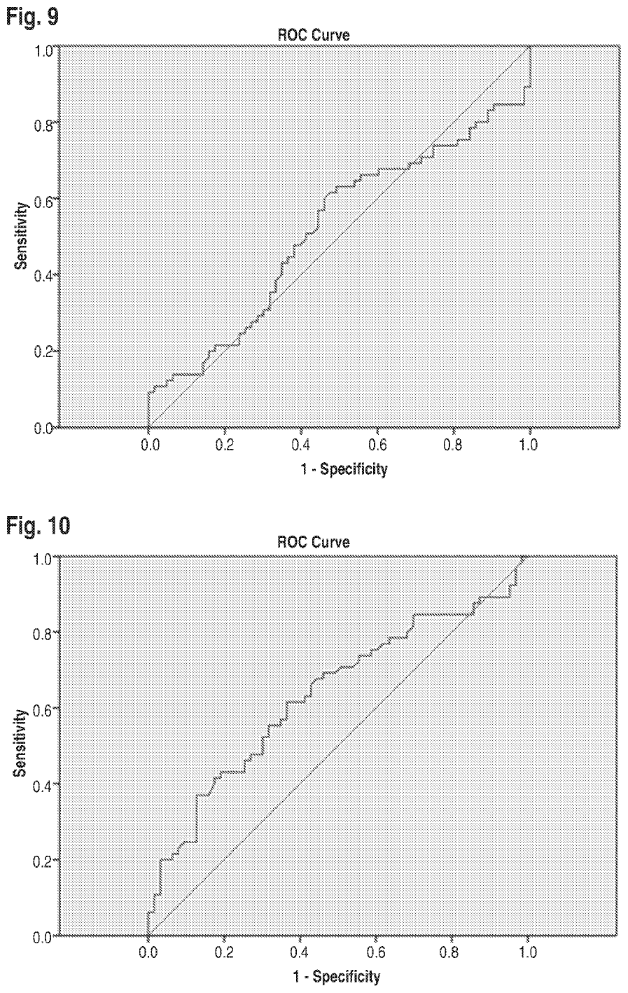

The results of the qPCR analysis are presented in Table 1, FIG. 1-3, and FIG. 8-10. The data revealed that each of the CDO1, PTGER4, HOXA9, SHOX2, SP9, and ZNF781 genes is able to distinguish malignant from benign lung nodules.

TABLE 1
Specificity and sensitivity of each biomarker in blood

| | CDO1 | PTGER4 | HOXA9 | SHOX2 | SP9 | ZNF781 |
|---|---|---|---|---|---|---|
| Sensitivity (%) | 46.6 | 39.4 | 26.5 | 33.8 | 60.0 | 61.5 |
| Specificity (%) | 90.3 | 94.9 | 80 | 89.1 | 54.0 | 63.5 |

### Example 3: Detection of Malignant Lung Nodule by Methylation Level of a Combination of at Least Two Genes Selected from CDO1, PTGER4, HOXA9, SHOX2, SP9, and ZNF781

The data collected in Example 2 was further analyzed to determine whether detection of methylation in combinations of the CDO1, PTGER4, HOXA9, SHOX2, SP9, and ZNF781 genes improves the ability to distinguish malignant from benign lung nodules. The area under the receiver operating characteristic curve (ROC) was calculated as an accuracy index using the 2^{-Δ}^{^{Ct}} values for evaluating the diagnostic performance of the selected specific genes. All data analyses were performed with GraphPad Prism.

### Results

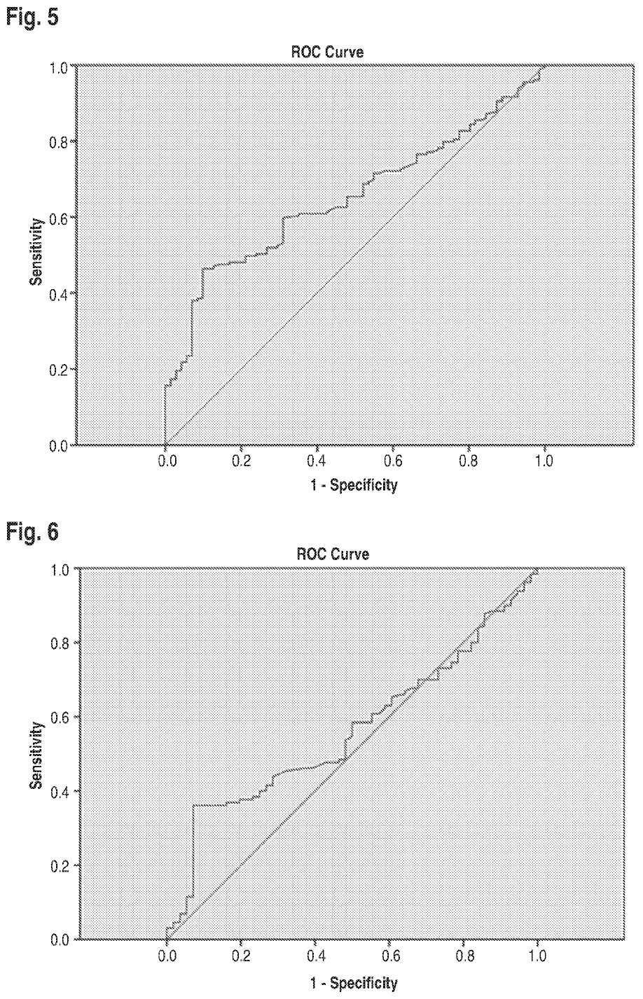

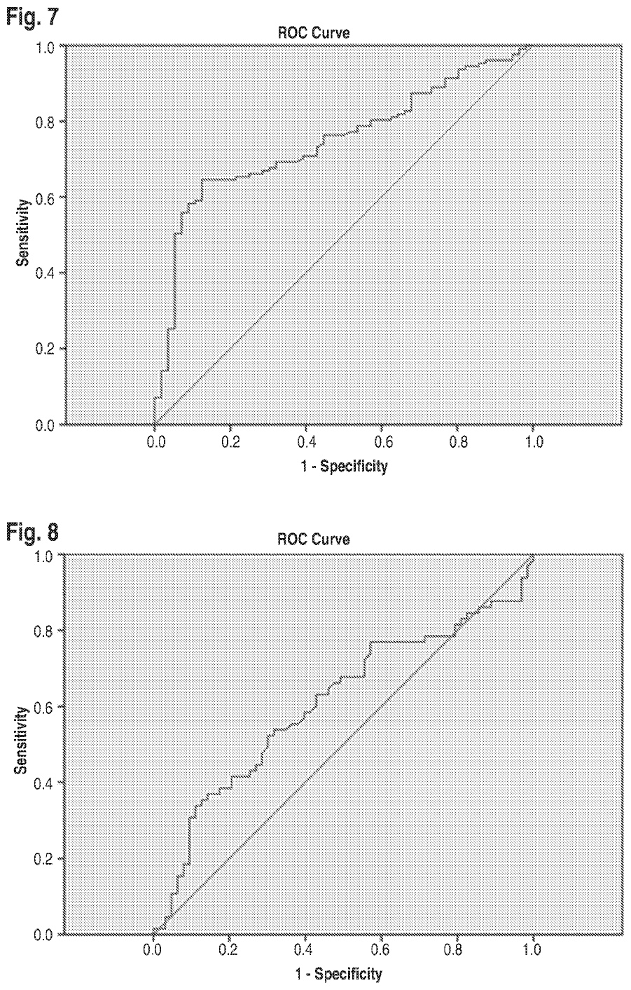

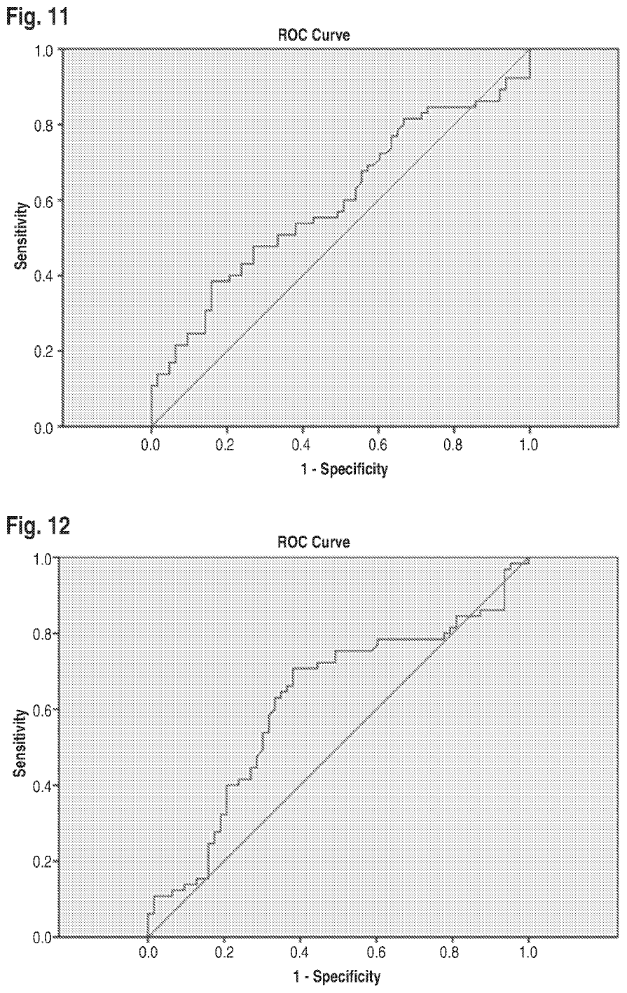

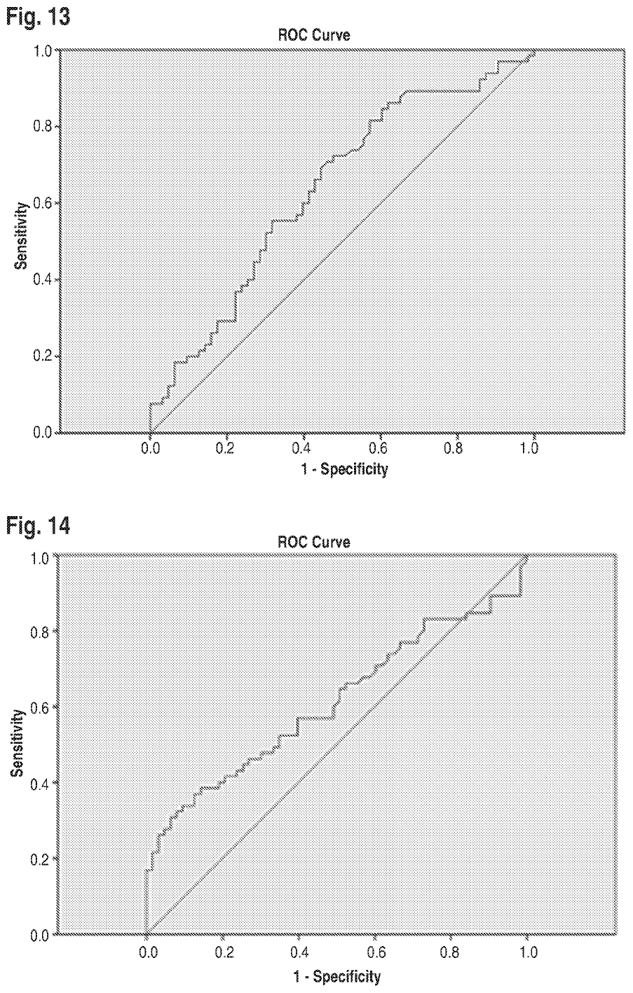

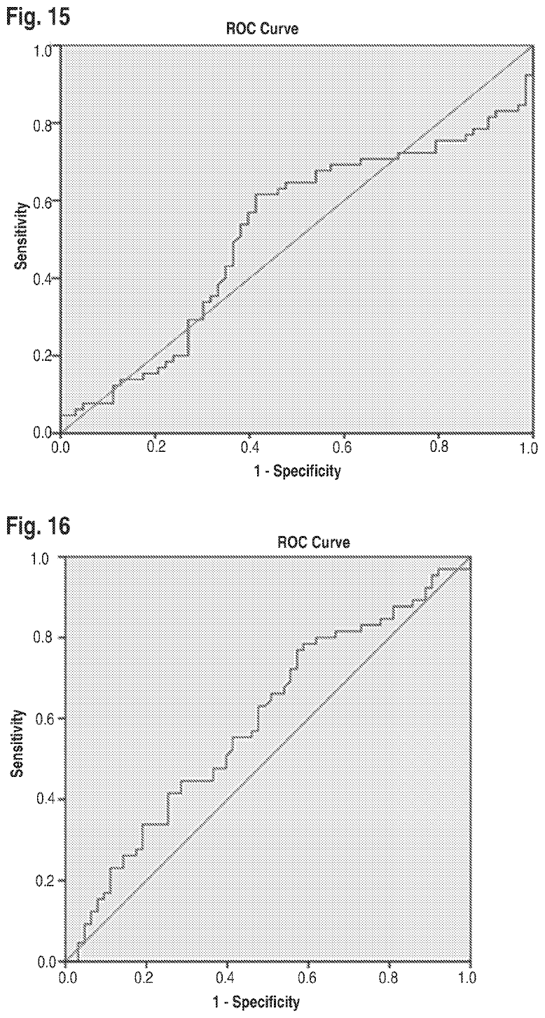

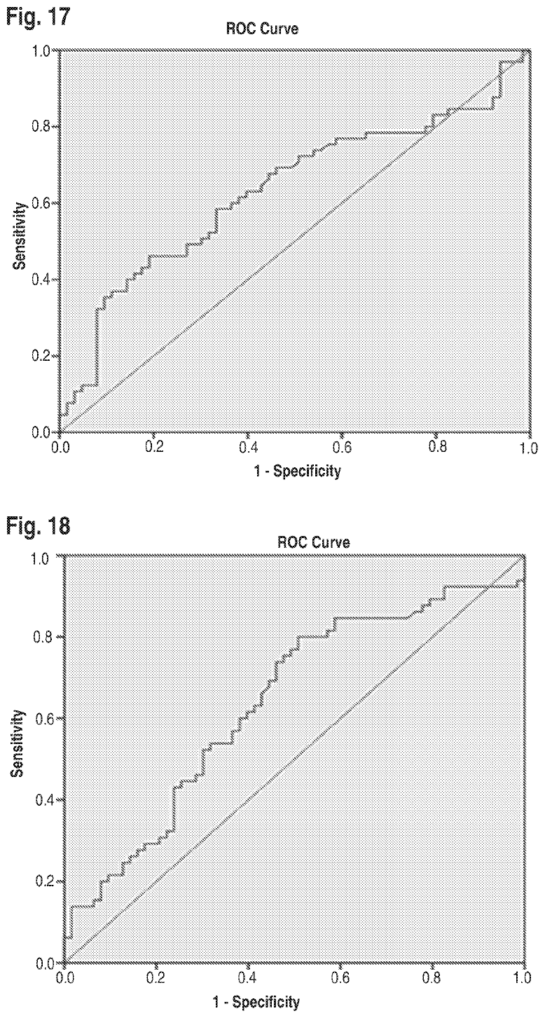

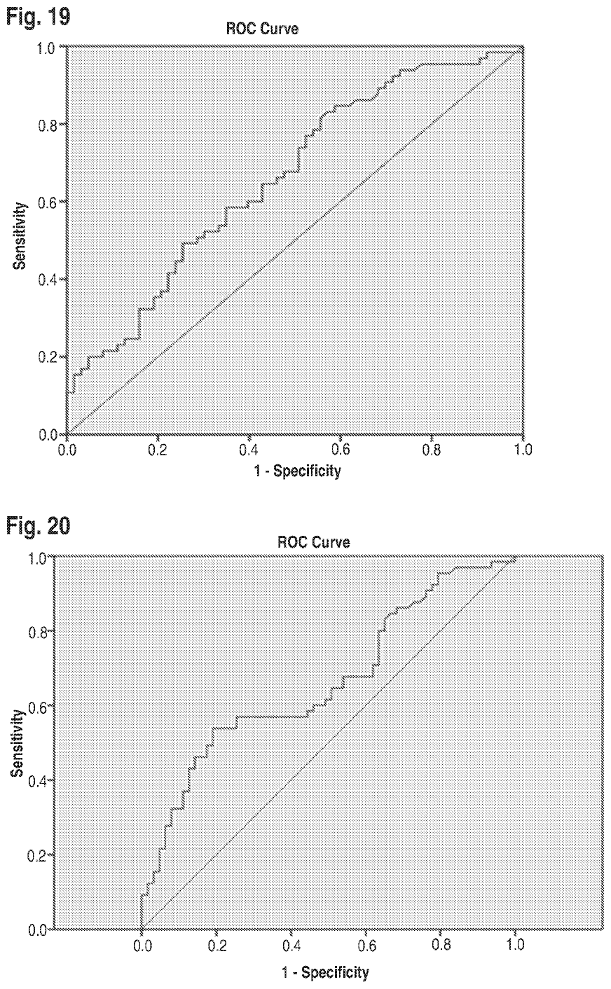

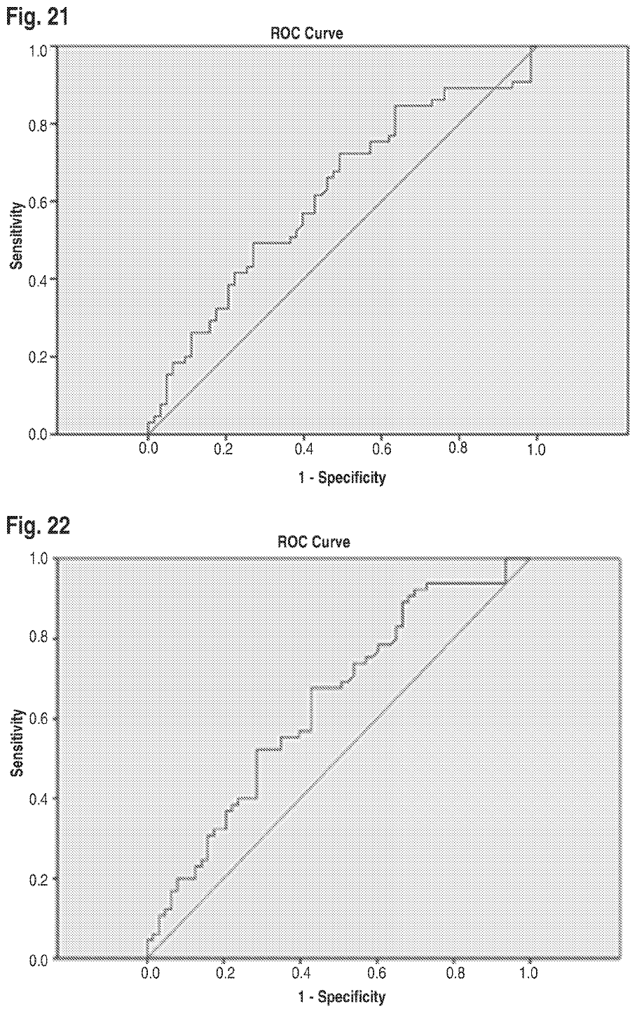

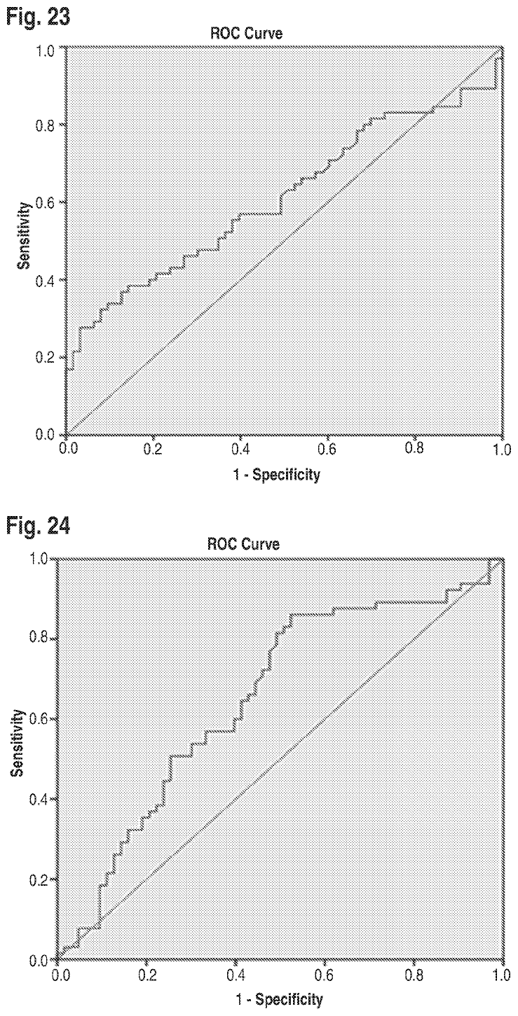

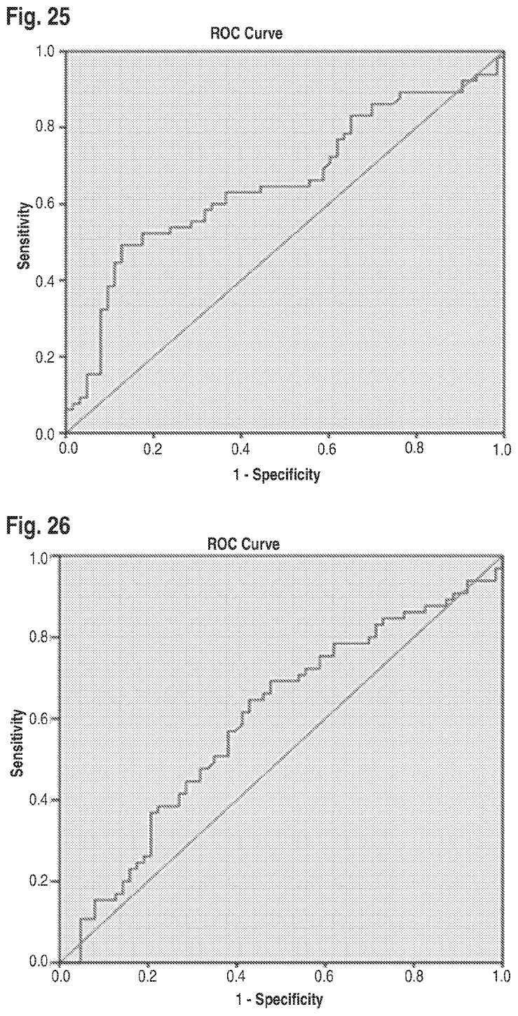

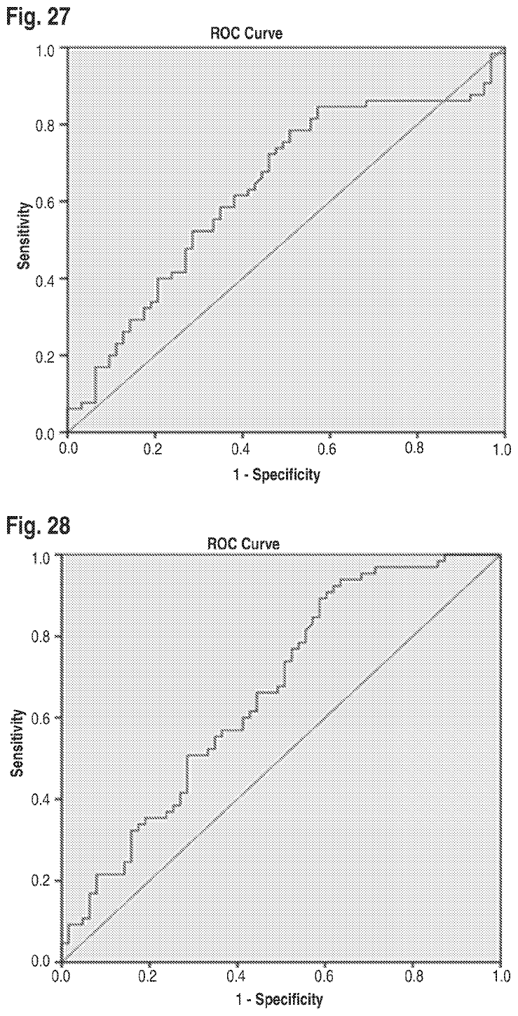

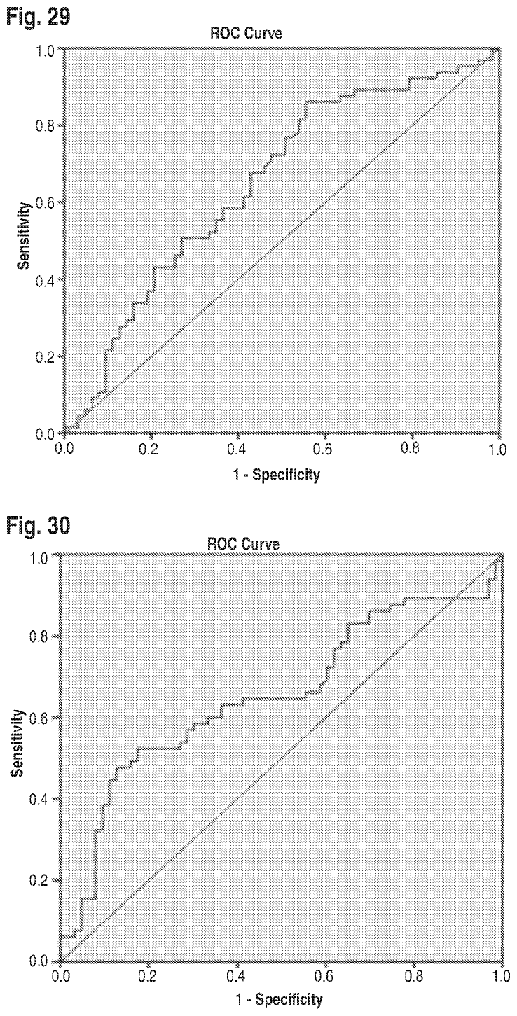

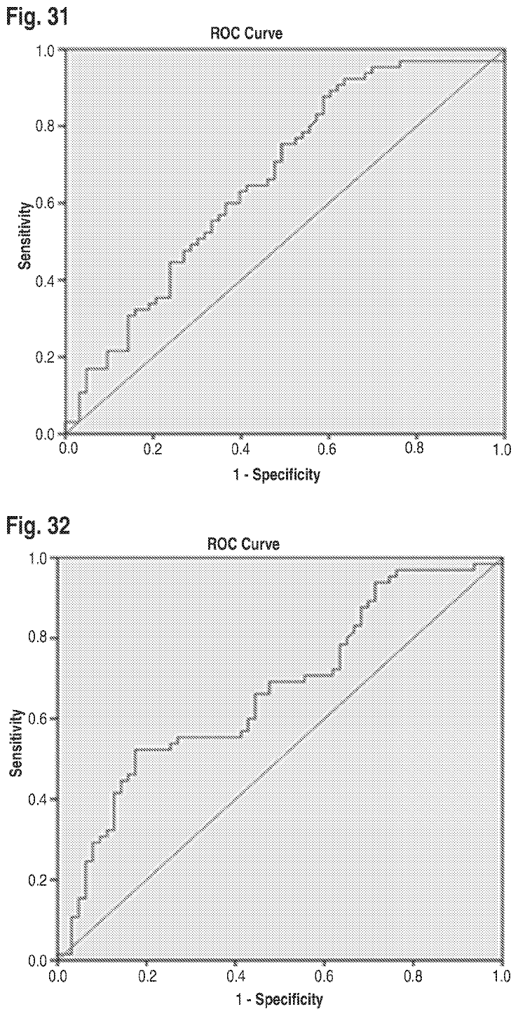

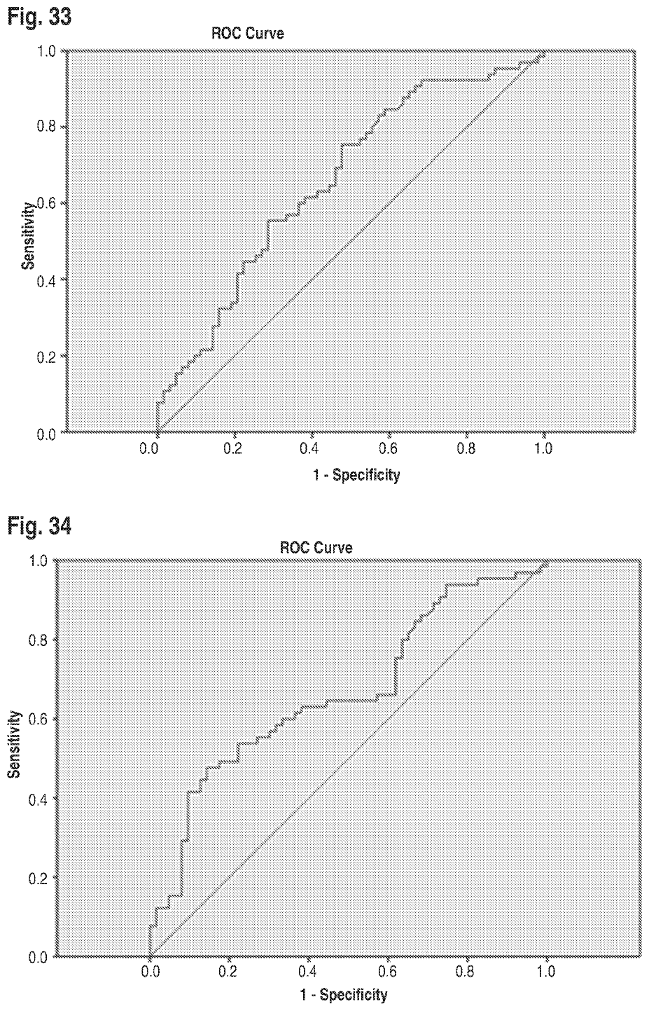

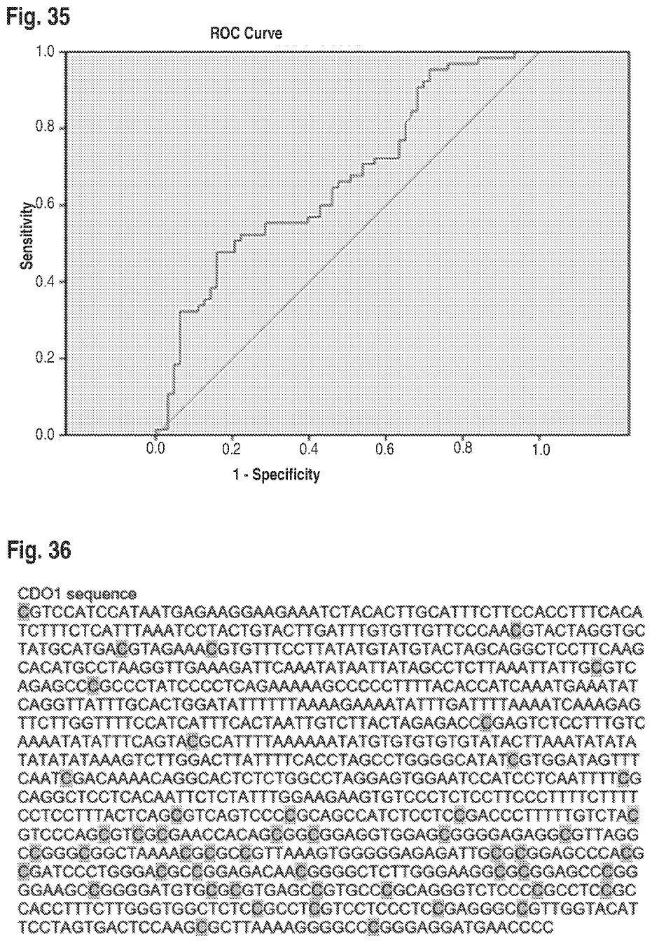

The further analysis are presented in Table 2, FIG. 4-7, and FIG. 11-35. The analysis showed that the combination yield better sensitivity and specificity than the individual genes alone. The analysis further revealed that the combination of CDO1 and PTGER4 yields the best sensitivity and specificity in the differentiation of malignant lung nodules from benign lung nodules. Adding HOXA9 to the combination of CDO1 and PTGER4 does not improve sensitivity and specificity.

TABLE 2
Results of the combined analysis of
at least two biomarkers in blood.

| | Sensitivity (%) | Specificity (%) |
|---|---|---|
| 1 + 2 | 38.5 | 84.1 |
| 1 + 3 | 70.8 | 61.9 |
| 1 + 4 | 69.2 | 55.6 |
| 1 + 5 | 32.3 | 92.2 |
| 2 + 3 | 61.5 | 58.7 |
| 2 + 4 | 76.9 | 42.9 |
| 2 + 5 | 46.2 | 81.0 |
| 3 + 4 | 64.6 | 87.5 |
| 4 + 6 | 59.8 | 69.0 |
| 3 + 6 | 43.8 | 71.4 |
| 3 + 5 | 80.0 | 49.2 |
| 4 + 5 | 83.1 | 42.9 |
| 1 + 2 + 3 | 72.3 | 50.8 |
| 1 + 2 + 4 | 67.7 | 57.1 |
| 1 + 2 + 5 | 27.7 | 96.8 |
| 1 + 3 + 4 | 86.2 | 47.6 |
| 1 + 3 + 5 | 49.2 | 87.3 |
| 1 + 4 + 5 | 53.8 | 83.0 |
| 2 + 3 + 4 | 64.6 | 57.1 |
| 2 + 3 + 5 | 78.5 | 49.2 |
| 2 + 4 + 5 | 55.4 | 71.5 |
| 3 + 4 + 5 | 89.2 | 41.3 |
| 3 + 4 + 6 | 64.6 | 87.5 |
| 1 + 2 + 3 + 4 | 86.2 | 44.4 |
| 1 + 2 + 3 + 5 | 47.7 | 87.3 |
| 1 + 2 + 4 + 5 | 47.7 | 85.7 |
| 1 + 3 + 4 + 5 | 47.7 | 84.1 |
| 2 + 3 + 4 + 5 | 87.7 | 41.3 |
| 1 + 2 + 3 + 4 + 5 | 52.3 | 82.5 |

1: SHOX2;
2: SP9;
3: PTGER4;
4: CDO1;
5: ZNF781;
6. HOXA9

TABLE 3
Sequences of primers and probes

| | | |
|---|---|---|
| SEQ ID NO: 1 | CDO1 forward | ACGTTTT |
| | primer | TTTTCGT |
| | | TTTATTT |
| | | TCGTCG |
| SEQ ID NO: 2 | CDO1 reverse | TCCTCCG |
| | primer | ACCCTTT |
| | | TTATCTA |
| | | CG |
| SEQ ID NO: 3 | CDO1 | GTGGTTC |
| | probe | GCGACGT |
| | | TGGGACG |
| | | TA |
| SEQ ID NO: 4 | PTGER4 forward | TTATTTT |
| | primer | CGGGGTT |
| | | AATTCGT |
| | | TC |
| SEQ ID NO: 5 | PTGER4 reverse | ATAAACA |
| | primer | TCACCGC |
| | | CGAAATA |
| SEQ ID NO: 6 | PTGER4 | TTGAGTT |
| | probe | TCGATCG |
| | | GTTGAAT |
| | | AGTTTAG |
| | | TGA |
| SEQ ID NO: 7 | HOXA9 forward | TATCGCG |
| | primer | GGTGTAG |
| | | GTTTT |
| SEQ ID NO: 8 | HOXA9 reverse | AAACGAA |
| | primer | TTTAAAA |
| | | ATTACCC |
| | | GAACG |
| SEQ ID NO: 9 | HOXA9 | ATAGTTA |
| | probe | TTCGTTT |
| | | GGAGCGA |
| | | GGGGCG |
| SEQ ID NO: 10 | SHOX2 forward | AATCGAG |
| | primer | GATCGCG |
| | | AATATTT |
| | | C |
| SEQ ID NO: 11 | SHOX2 reverse | TCGTAAA |
| | primer | AACGCAA |
| | | ACTCGAC |
| SEQ ID NO: 12 | SHOX2 | TTGAATT |
| | probe | TGTTGAT |
| | | TTTTGTG |
| | | GGTTAAG |
| | | CGTTTAG |
| | | G |
| SEQ ID NO: 13 | SP9 forward | TATTTAG |
| | primer | GTTGAGT |
| | | TTAGTCG |
| | | GC |
| SEQ ID NO: 14 | SP9 reverse | CGAAAAA |
| | primer | ACTACTT |
| | | AAATCCC |
| | | GC |
| SEQ ID NO: 15 | SP9 | AGTTTGG |
| | probe | AGTTCGT |
| | | TCGGTAC |
| | | GAGC |
| SEQ ID NO: 16 | ZNF781 forward | GTCGTTG |
| | primer | GTATAAG |
| | | TTGCGTT |
| | | C |
| SEQ ID NO: 17 | ZNF781 reverse | AAACTAT |
| | primer | AACCCGA |
| | | TAAATCC |
| | | GC |
| SEQ ID NO: 18 | ZNF781 | AGACGTG |
| | probe | GGAGCGT |
| | | TTTTTTG |
| | | TTTTTCG |

1. A method comprising
measuring the methylation level of at least two genes in a sample from a subject, wherein the at least two genes are selected from CDO1, PTGER4, HOXA9, SHOX2, SP9, and ZNF781, and
determining if the at least two genes are hypermethylated,
wherein detection of hypermethylation of the least two genes indicates that the subject has at least one malignant lung nodule and/or lung cancer.

2. A method comprising
measuring the methylation level of at least two genes in a sample from a subject, wherein the at least two genes are selected from CDO1, PTGER4, HOXA9, SHOX2, SP9, and ZNF781,
determining if the at least two genes are hypermethylated,
diagnosing the subject as having at least one malignant lung nodule and/or lung cancer when hypermethylation of the at least two genes is detected, and
administering an effective amount of at least one lung cancer treatment to the subject diagnosed as having at least one malignant lung nodule and/or lung cancer.

3. The method of claim 2, wherein the lung cancer treatment is selected from surgery, chemotherapy, radiation therapy, immunotherapy, and targeted drug therapy.

4. The method of claim 1, wherein the sample is a blood sample, a sputum sample, a sample collected from bronchial washing, a sample collected from bronchial brushing, a urine sample, or a saliva sample.

5. The method of claim 1, comprising:
(a) collecting blood sample from the subject;
(b) isolating plasma from the blood sample;
(c) extracting DNA from the isolated plasma;
(d) measuring the methylation level of at least two genes in the extracted DNA, wherein the at least two genes are selected from CDO1, PTGER4, HOXA9, SHOX2, SP9, and ZNF781; and
(e) determining if the at least two genes are hypermethylated, wherein detection of hypermethylation of the at least two genes indicates that the subject has at least one malignant lung nodule and/or lung cancer;
wherein the methylation level is measured by
(1) converting unmethylated cytosine in the DNA in step (c) to uracil while leaving methylated cytosine as cytosine, and
(2) measuring the level of conversion of unmethylated cytosine to uracil.

6. The method of claim 5, wherein the unmethylated cytosine in the DNA is converted to uracil by bisulfite treatment or enzyme treatment.

7. The method of claim 5, wherein the measuring is carried out by real-time polymerase chain reaction (PCR), sequencing, or microarray.

8. The method of claim 7, wherein the PCR is a methylation-specific quantitative real-time PCR.

9. The method of claim 8, wherein the methylation-specific quantitative real-time PCR uses a forward primer comprising SEQ ID NO: 1, a reverse primer comprising SEQ ID NO: 2, and a probe comprising SEQ ID NO: 3.

10. The method of claim 8, wherein the methylation-specific quantitative real-time PCR uses a forward primer comprising SEQ ID NO: 4, a reverse primer comprising SEQ ID NO: 5, and a probe comprising SEQ ID NO: 6.

11. The method of claim 8, wherein the methylation-specific quantitative real-time PCR uses a forward primer comprising SEQ ID NO: 7, a reverse primer comprising SEQ ID NO: 8, and a probe comprising SEQ ID NO: 9.

12. The method of claim 8, wherein the methylation-specific quantitative real-time PCR uses a forward primer comprising SEQ ID NO: 10, a reverse primer comprising SEQ ID NO: 11, and a probe comprising SEQ ID NO: 12.

13. The method of claim 8, wherein the methylation-specific quantitative real-time PCR uses a forward primer comprising SEQ ID NO: 13, a reverse primer comprising SEQ ID NO: 14, and a probe comprising SEQ ID NO: 15.

14. The method of claim 8, wherein the methylation-specific quantitative real-time PCR uses a forward primer comprising SEQ ID NO: 16, a reverse primer comprising SEQ ID NO: 17, and a probe comprising SEQ ID NO: 18.

15. The method of claim 1, wherein the methylation level is measured by the methylation-specific high-resolution melting, pyrosequencing, nanopore long-read technology, or methylation-specific restriction enzyme digestion.

16. The method of claim 1, wherein the lung cancer is a non-small cell lung cancer or a small cell lung cancer.

17. The method of claim 1, wherein the subject has previously been determined as having at least one lung nodule.

18. The method of claim 1, wherein the subject is from a population with high risk of getting lung cancer.

19. A polynucleotide having a sequence of any one of SEQ ID NOs: 1-18.

20. A kit for conducting methylation-specific quantitative real-time PCR of CDO1, comprising a forward primer of SEQ ID NO: 1, a reverse primer of SEQ ID NO: 2, and a probe of SEQ ID NO: 3, optionally further comprising bisulfite.

21. A kit for conducting methylation-specific quantitative real-time PCR of PTGER4, comprising a forward primer of SEQ ID NO: 4, a reverse primer of SEQ ID NO: 5, and a probe of SEQ ID NO: 6, optionally further comprising bisulfite.

22. A kit for conducting methylation-specific quantitative real-time PCR of HOXA9, comprising a forward primer of SEQ ID NO: 7, a reverse primer of SEQ ID NO: 8, and a probe of SEQ ID NO: 9, optionally further comprising bisulfite.

23. A kit for conducting methylation-specific quantitative real-time PCR of SHOX2, comprising a forward primer of SEQ ID NO: 10, a reverse primer of SEQ ID NO: 11, and a probe of SEQ ID NO: 12, optionally further comprising bisulfite.

24. A kit for conducting methylation-specific quantitative real-time PCR of SP9, comprising a forward primer of SEQ ID NO: 13, a reverse primer of SEQ ID NO: 14, and a probe of SEQ ID NO: 15, optionally further comprising bisulfite.

25. A kit for conducting methylation-specific quantitative real-time PCR of ZNF781, comprising a forward primer of SEQ ID NO: 16, a reverse primer of SEQ ID NO: 17, and a probe of SEQ ID NO: 18, optionally further comprising bisulfite.

26. (canceled)

27. A kit for determining whether at least one lung nodule found in a subject is malignant, comprising bisulfite, and reagents for conducting methylation-specific quantitative real-time PCR of at least two of the genes selected from CDO1, PTGER4, HOXA9, SHOX2, SP9, and ZNF781,
wherein the reagents for conducting methylation-specific quantitative real-time PCR of CDO1 comprise a CDO1 forward primer, a CDO1 reverse primer, and a CDO1 probe,
wherein the reagents for conducting methylation-specific quantitative real-time PCR of PTGER4 comprise a PTGER4 forward primer, a PTGER4 reverse primer, and a PTGER4 probe,
wherein the reagents for conducting methylation-specific quantitative real-time PCR of HOXA9 comprise a HOXA9 forward primer, a HOXA9 reverse primer, and a HOXA9 probe,
wherein the reagents for conducting methylation-specific quantitative real-time PCR of SHOX2 comprise a SHOX2 forward primer, a SHOX2 reverse primer, and a SHOX2 probe,
wherein the reagents for conducting methylation-specific quantitative real-time PCR of SP9 comprise a SP9 forward primer, a SP9 reverse primer, and a SP9 probe,
wherein the reagents for conducting methylation-specific quantitative real-time PCR of ZNF781 comprise a ZNF781 forward primer, a ZNF781 reverse primer, and a ZNF781 probe.

28. The kit of claim 27, further comprising at least two of the kits selected from:
a kit for conducting methylation-specific quantitative real-time PCR of CDO1, comprising a forward primer of SEQ ID NO: 1, a reverse primer of SEQ ID NO: 2, and a probe of SEQ ID NO: 3;
a kit for conducting methylation-specific quantitative real-time PCR of PTGER4, comprising a forward primer of SEQ ID NO: 4, a reverse primer of SEQ ID NO: 5, and a probe of SEQ ID NO: 6;
a kit for conducting methylation-specific quantitative real-time PCR of HOXA9, comprising a forward primer of SEQ ID NO: 7, a reverse primer of SEQ ID NO: 8, and a probe of SEQ ID NO: 9;
a kit for conducting methylation-specific quantitative real-time PCR of SHOX2, comprising a forward primer of SEQ ID NO: 10, a reverse primer of SEQ ID NO: 11, and a probe of SEQ ID NO: 12;
a kit for conducting methylation-specific quantitative real-time PCR of SP9, comprising a forward primer of SEQ ID NO: 13, a reverse primer of SEQ ID NO: 14, and a probe of SEQ ID NO: 1; and
a kit for conducting methylation-specific quantitative real-time PCR of ZNF781, comprising a forward primer of SEQ ID NO: 16, a reverse primer of SEQ ID NO: 17, and a probe of SEQ ID NO: 18.

29. The method of claim 2, wherein the sample is a blood sample, a sputum sample, a sample collected from bronchial washing, a sample collected from bronchial brushing, a urine sample, or a saliva sample.

30. The method of claim 2, comprising:
(a) collecting blood sample from the subject;
(b) isolating plasma from the blood sample;
(c) extracting DNA from the isolated plasma;
(d) measuring the methylation level of at least two genes in the extracted DNA, wherein the at least two genes are selected from CDO1, PTGER4, HOXA9, SHOX2, SP9, and ZNF781;
(e) determining if the at least two genes are hypermethylated, wherein detection of hypermethylation of the at least two genes indicates that the subject has at least one malignant lung nodule and/or lung cancer;
(f) diagnosing the subject as having at least one malignant lung nodule and/or lung cancer when hypermethylation of the at least two genes is detected; and
(g) administering an effective amount of at least one lung cancer treatment to the subject diagnosed as having at least one malignant lung nodule and/or lung cancer.
wherein the methylation level is measured by
(1) converting unmethylated cytosine in the DNA in step (c) to uracil while leaving methylated cytosine as cytosine, and
(2) measuring the level of conversion of unmethylated cytosine to uracil.

31. The method of claim 30, wherein the unmethylated cytosine in the DNA is converted to uracil by bisulfite treatment or enzyme treatment.

32. The method of claim 30, wherein the measuring is carried out by real-time polymerase chain reaction (PCR), sequencing, or microarray.

33. The method of claim 32, wherein the PCR is a methylation-specific quantitative real-time PCR.

34. The method of claim 33, wherein the methylation-specific quantitative real-time PCR uses a forward primer comprising SEQ ID NO: 1, a reverse primer comprising SEQ ID NO: 2, and a probe comprising SEQ ID NO: 3.

35. The method of claim 33, wherein the methylation-specific quantitative real-time PCR uses a forward primer comprising SEQ ID NO: 4, a reverse primer comprising SEQ ID NO: 5, and a probe comprising SEQ ID NO: 6.

36. The method of claim 33, wherein the methylation-specific quantitative real-time PCR uses a forward primer comprising SEQ ID NO: 7, a reverse primer comprising SEQ ID NO: 8, and a probe comprising SEQ ID NO: 9.

37. The method of claim 33, wherein the methylation-specific quantitative real-time PCR uses a forward primer comprising SEQ ID NO: 10, a reverse primer comprising SEQ ID NO: 11, and a probe comprising SEQ ID NO: 12.

38. The method of claim 33, wherein the methylation-specific quantitative real-time PCR uses a forward primer comprising SEQ ID NO: 13, a reverse primer comprising SEQ ID NO: 14, and a probe comprising SEQ ID NO: 15.

39. The method of claim 33, wherein the methylation-specific quantitative real-time PCR uses a forward primer comprising SEQ ID NO: 16, a reverse primer comprising SEQ ID NO: 17, and a probe comprising SEQ ID NO: 18.

40. The method of claim 2, wherein the methylation level is measured by the methylation-specific high-resolution melting, pyrosequencing, nanopore long-read technology, or methylation-specific restriction enzyme digestion.

41. The method of claim 2, wherein the lung cancer is a non-small cell lung cancer or a small cell lung cancer.

42. The method of claim 2, wherein the subject has previously been determined as having at least one lung nodule.

43. The method of claim 2, wherein the subject is from a population with high risk of getting lung cancer.
